SEQUENCE LISTING (4 sequences)

SEQ ID NO: 1 - DNA, 33 bp, Artificial Sequence
  Description of Artificial Sequence Synthetic oligonucleotide
tttgcatggctacattcagatgacacatgcaaa

SEQ ID NO: 2 - DNA, 33 bp, Artificial Sequence
  Description of Artificial Sequence Synthetic oligonucleotide
tttgcatcgtacgcggaatacttcgaatgcaaa

SEQ ID NO: 3 - DNA, 33 bp, Artificial Sequence
  Description of Artificial Sequence Synthetic oligonucleotide
tttgcatttctccgaacgtgtcacgtatgcaaa

SEQ ID NO: 4 - DNA, 19 bp, Unknown
  Description of Unknown MRP1 target sequence
gtgtcatctgaatgtagcc

(19) United States
(12) Patent Application Publication
(10) Pub. No.: US 2016/0243254 A1
(43) Pub. Date: Aug. 25, 2016

(54) Theranostic Nanoprobes for Overcoming Cancer Multidrug Resistance and Methods

(71) Applicant: Massachusetts Institute of Technology, Cambridge, MA (US)

(72) Inventors: Natalie Artzi, Brookline, MA (US); João Conde, Cambridge, MA (US); Nuria Oliva, Somerville, MA (US)

(21) Appl. No.: 15/047,902

(22) Filed: Feb. 19, 2016

Related U.S. Application Data
(60) Provisional application No. 62/118,101, filed on Feb. 19, 2015.

(51) Int. Cl. A61K 47/48; A61K 31/513; A61K 47/34; A61K 47/36; A61K 49/00; A61K 9/06

(52) U.S. Cl. CPC A61K 47/48884; A61K 47/48092; A61K 49/0093; A61K 49/0052; A61K 49/0032; A61K 49/0021; A61K 9/06; A61K 47/34; A61K 47/36; A61K 31/513

(57) ABSTRACT

Theranostic nanoprobes are provided for overcoming cancer multidrug resistance, and methods for treating biological tissue, including cancerous tissue. The theranostic nanoprobes may include gold nanoparticles functionalized with DNA-hairpin. The DNA-hairpin may be configured to hybridize to a complementary target, which may silence or lessen the multidrug resistance of cancer cells. The theranostic nanoprobes may be configured to release a chemotherapeutic agent upon hybridization of the DNA-hairpin to a target molecule.

### CROSS-REFERENCE TO RELATED APPLICATION

This application claims priority to U.S. Provisional Patent Application No. 62/118,101, filed Feb. 19, 2015, which is incorporated herein by reference.

### BACKGROUND

Multidrug resistance (MDR) in cancer cells can substantially limit the success of chemotherapy. MDR in cancer is a phenomenon whereby cancer cells gain the capacity to develop cross resistance and survive a variety of structurally and functionally unrelated drugs.

The most common MDR mechanisms occur by the expression of one or more energy-dependent transporters, which can result in an increased efflux of the cytotoxic drugs from the cancer cells, thus lowering their intracellular concentrations. The phosphoglycoprotein multidrug resistance protein 1 (MRP1-ABCC1) is often associated with resistance to a broad spectrum of anticancer drugs and belongs to the ATP-binding cassette (ABC) superfamily of proteins as energy-dependent efflux pumps.

The ABC transporters are essential not only to breast cancer MDR but also other types of cancer, such as non-small cell lung cancer, lung cancer, and rectal cancer. In fact, increased ABC expression levels have been shown to correlate with decreased response to various chemotherapy drugs, such as 5-fluorouracil (5-FU), and a decline in overall survival.

5-FU is widely used in cancer therapy as it has the capacity to interfere with nucleoside metabolism and result in DNA and RNA synthesis disorders and dysfunction, leading to cytotoxicity and cell death. Based on the American Cancer Society guidelines, 5-FU is used to treat a range of cancerous diseases, including colon and rectal cancer, breast cancer, gastrointestinal cancers, including anal, esophageal, pancreas and gastric (stomach), head and neck cancer, and ovarian cancer. Although 5-FU is well known for treating head and neck cancer, it also has been extensively used for treating breast cancer. For decades, 5-FU has been used in combination with other antineoplastic agents or as a single agent in the adjuvant and palliative treatment of advanced breast cancer.

Nevertheless, the overall response to this drug remains only 15% due to resistance mechanisms. Therefore, despite the remarkable progress in chemotherapeutic drug development in the past decade, approximately 70% of cancer patients do not respond to chemotherapy, and have a five-year survival rate of 10-30%.

The development of nanoscale devices has provided several breakthroughs in cancer diagnostics and therapy, including drug delivery. Nanoparticles can be excellent tumor-targeting vehicles due to enhanced permeability and retention (EPR) in the tumor microenvironment, which may have defective vasculature and poor lymphatic drainage. Therefore, it is possible that nanoparticles can be harnessed to alter MDR mechanisms. Although some nanomaterials have been employed to help overcome MDR by increasing drug retention in cancer cells and hampering cancer progression, a platform that senses and inhibits MDR prior to selective drug release has not been reported, but would be desirable.

Platforms that are able to improve antitumor efficacy are needed, including those that address the difficulties associated with multidrug resistance in cancer cells.

### BRIEF SUMMARY

Provided herein are theranostic nanoprobes. In embodiments, the theranostic nanoprobes comprise a gold nanoparticle functionalized with at least one DNA-hairpin, and a chemotherapeutic agent intercalated in the at least one DNA-hairpin, wherein the at least one DNA-hairpin is configured to hybridize to a complementary target in a cancer cell. The complementary target, in one embodiment, is MRP1 mRNA. In particular embodiments, the theranostic nanoprobes further comprise a hydrogel in which the gold nanoparticle is embedded. The hydrogel may include a dendrimer and a polymer.

In embodiments, the theranostic nanoprobes provided herein comprise [1] a gold nanoparticle functionalized with at least one DNA-hairpin labeled with a fluorophore, and at least one anchor labeled with a quencher, and [2] a chemotherapeutic agent intercalated in the at least one DNA-hairpin, wherein the at least one DNA-hairpin is configured to hybridize to a complementary target in a cancer cell. The complementary target, in embodiments, is MRP1 mRNA. The anchor may comprise a DNA-oligonucleotide. In one embodiment, the gold nanoparticle is further functionalized with a spacer comprising PEG. The spacer comprising PEG may be derived from α-Mercapto-ω-carboxy PEG. In some embodiments, the theranostic nanoprobes are embedded in a hydrogel. The hydrogel, in particular embodiments, comprises a dendrimer and a polymer.

Methods for treating a biological tissue, including tumor tissue, also are provided. In embodiments, the methods comprise providing a hydrogel comprising an embedded theranostic nanoprobe; and contacting the biological tissue with the hydrogel; wherein the embedded theranostic nanoprobe comprises [1] a gold nanoparticle functionalized with at least one DNA-hairpin labeled with a fluorophore, and at least one anchor labeled with a quencher; and [2] a chemotherapeutic agent intercalated in the at least one DNA-hairpin; wherein the at least one DNA-hairpin is configured to hybridize to a complementary target in a cancer cell, e.g., of the tumor tissue. The at least one anchor may include DNA-oligonucleotide.

In other embodiments, the methods comprise providing a first solution comprising a polymer component, wherein the polymer component comprises a polymer having three or more aldehyde groups; providing a second solution comprising a dendrimer component, wherein the dendrimer component comprises a dendrimer having at least 2 branches with one or more surface groups; wherein at least one of the first solution and second solution comprises a theranostic nanoprobe; and combining the first and second solutions together to produce a hydrogel and contacting one or more biological tissues with the hydrogel; wherein the embedded theranostic nanoprobe comprises [1] a gold nanoparticle functionalized with at least one DNA-hairpin labeled with a fluorophore, and at least one anchor labeled with a quencher; and [2] a chemotherapeutic agent intercalated in the at least one DNA-hairpin; wherein the at least one DNA-hairpin is configured to hybridize to a complementary target in a cancer cell. The at least one anchor may include DNA-oligonucleotide.

In the methods provided herein, contacting a biological tissue with a hydrogel comprises, in one embodiment, applying the hydrogel on a surface of the biological tissue. In another embodiment, contacting the biological tissue with the hydrogel comprises injecting the hydrogel into the biological tissue.

### BRIEF DESCRIPTION OF THE DRAWINGS

FIG. 1A depicts one embodiment of a dark-gold nanobeacon designed to sense and overcome cancer MDR.

FIG. 1B depicts the fluorescent intensity change of the near-infrared dye Quasar® 705 (0.5 μM) after reaction with one embodiment of dark-gold nanobeacons at different concentrations from 0 to 10 nM (excitation wavelength=675 nm).

FIG. 1C depicts fluorescence emission spectra of 5-FU (1 mM) after incubation with 2.5 nM of one embodiment of dark-gold nanobeacons-Q705 for different periods of time (0-120 minutes).

FIG. 1D depicts Quasar® 705 emission spectra after hybridization of one embodiment of anti-MRP1 nanobeacons and nonsense nanobeacons with increasing amounts of a complementary and noncomplementary ssDNA target (0-5 μM).

FIG. 1E depicts 5-FU emission spectra after hybridization of one embodiment of anti-MRP1 nanobeacons and nonsense nanobeacons with increasing amounts of a complementary and noncomplementary ssDNA target (0-5 μM).

FIG. 2 depicts a flow cytometry analysis comparing one embodiment of anti-Luc nanobeacon-treated cells and one embodiment of anti-MRP1 nanobeacon-treated cells to nanobeacon nonsense.

FIG. 3A depicts an evaluation of the change in tumor size as a function of time after treatment with one embodiment of nanobeacon (n=5, ***P<0.005).

FIG. 3B depicts the signal of one embodiment of a nanobeacon probe as a function of time after treatment with nanobeacons (n=5, ***P<0.005).

FIG. 4A depicts two-dimensional structures of the sequences of DNA-hairpins for one embodiment of an anti-MRP1 nanobeacon, one embodiment of an anti-Luc nanobeacon, and a nonsense nanobeacon at 37° C.

FIG. 4B depicts normalized absorbance and emission spectra of one embodiment of a dark-gold nanobeacon that includes gold nanoparticles functionalized with a DNA-oligonucleotide labeled with a dark quencher, one embodiment of a free DNA-oligonucleotide labeled with Quasar® 705, and one embodiment of a free DNA-oligonucleotide labeled with BHQ2 dark quencher.

FIG. 4C depicts normalized absorbance and emission spectra of one embodiment of gold nanoparticles functionalized with a DNA-oligonucleotide labeled with Quasar® 705, and free drug 5-FU.

FIG. 5 depicts a comparison of the fluorescence of 5-FU (emission at 400 nm) intercalated in the nanobeacons, and the fluorescence of Quasar® 705 (emission at 705 nm) in the presence of a complementary and non-complementary target.

FIG. 6 depicts the results of an MTT assay for a resistant breast cancer cell line (MDA-MB-231) after continuous exposure to drug (5-FU), compared to parental cancer cells.

FIG. 7 depicts a plot of tumor size (mm³) against days after implantation of one embodiment of a hydrogel containing one embodiment of a theranostic nanoprobe.

FIG. 8 depicts a plot of relative MRP1 expression/GAPDH mRNA level versus the concentration of several embodiments of nanobeacons.

FIG. 9A depicts a plot of cell viability (%) for several embodiments of nanobeacons against incubation time (hours).

FIG. 9B depicts a plot of relative MRP1 expression/GAPDH mRNA level for several embodiments of nanobeacons against incubation time (hours).

FIG. 10 depicts a plot of hydrogel degradation versus days after implantation of one embodiment of a hydrogel included one of several embodiments of nanobeacons.

### DETAILED DESCRIPTION

Provided herein are theranostic nanoprobes that can be used to lessen or eliminate cross-resistance to many chemotherapeutic drugs. It has been demonstrated that embodiments of the theranostic nanoprobes provided herein were able to reduce about 90% of 5-FU drug-resistant tumors 14 days after implantation in breast cancer tumor bearing mice by silencing over 80% of MRP1 expression prior to drug release.

The theranostic nanoprobes provided herein may include an on/off molecular nanoswitch that is triggered by a target molecule. The target molecule may be a specific gene sequence, such as MRP1 mRNA. Therefore, the on/off molecular nanoswitch of the theranostic nanoprobes provided herein can be triggered by the increased expression ofMRP1 within a tumor, including the tumor tissue microenvironment. As a result, the theranostic nanoprobes advantageously can sense, and lessen or silence, MDR prior to local drug release, during local drug release, or a combination thereof.

The theranostic nanoprobes may include gold nanoparticles functionalized with a DNA-hairpin. Gold nanoparticles functionalized in this manner are sometimes referred to in the art as “nanobeacons” or “gold nanobeacons.” At least one chemotherapeutic agent may be intercalated in the DNA-hairpin. The DNA-hairpin also may be labeled with at least one fluorophore. Although labeling the DNA-hairpin with at least one fluorophore permits the theranostic nanoprobes to report certain events as described herein, labeling is not required to lessen or silence MDR, achieve drug release, or a combination thereof.

In embodiments, the on/off molecular nanoswitch is provided by the DNA-hairpin. The DNA-hairpin may be configured to hybridize to a target molecule, and, upon hybridization, the DNA-hairpin “opens,” thereby converting the molecular switch from “off” to “on.” The nanobeacons provided herein can be designed to open and release an intercalated chemotherapeutic agent only upon hybridization of the DNA-hairpin to a complementary target. When the complementary target, in certain embodiments, is MRP1 mRNA of a tumor, the hybridization of the DNA-hairpin lessens or silences the tumor's drug resistance prior to release of the intercalated chemotherapeutic drug, during the release of the intercalated chemotherapeutic drug, or a combination thereof.

Moreover, the theranostic nanoprobes provided herein may be configured to report on these events. In embodiments, the theranostic nanoprobes include a dark quencher and a fluorophore. A fluorophore, in embodiments, labels the DNA-hairpin, and a dark quencher is bonded to the theranostic nanoprobes via an anchor, which may include DNA-oligonucleotide. The fluorophore and quencher may be chosen so that the conformational reorganization that occurs due to hybridization restores the fluorescence emission of the nanobeacons, including the fluorophore. As used herein, a gold nanobeacon functionalized with an anchor labeled with a quencher is referred to as a “dark-gold nanobeacon” or “dark nanobeacon.”

The theranostic nanoprobes provided herein also may be functionalized with a spacer, such as a spacer comprising thio-polyethylene glycol (PEG)-COOH. The spacer may [1] impart stability to the theranostic nanoprobes provided herein, [2] ensure facile functionalization of the gold nanoparticle with the DNA-hairpins, anchors, including DNA-oligonucleotides, or a combination thereof, [3] ensure a desirable distribution of DNA-hairpins on the surfaces of the gold nanoparticles, or [4] a combination thereof.

In embodiments, the nanoprobes provided herein can be used to overcome MDR by silencing the multidrug resistance protein 1 (MRP1) prior to and/or during chemotherapeutic drug delivery in vivo with a single topical application.

In embodiments, the theranostic nanoprobes provided herein are embedded in a hydrogel prior to use.

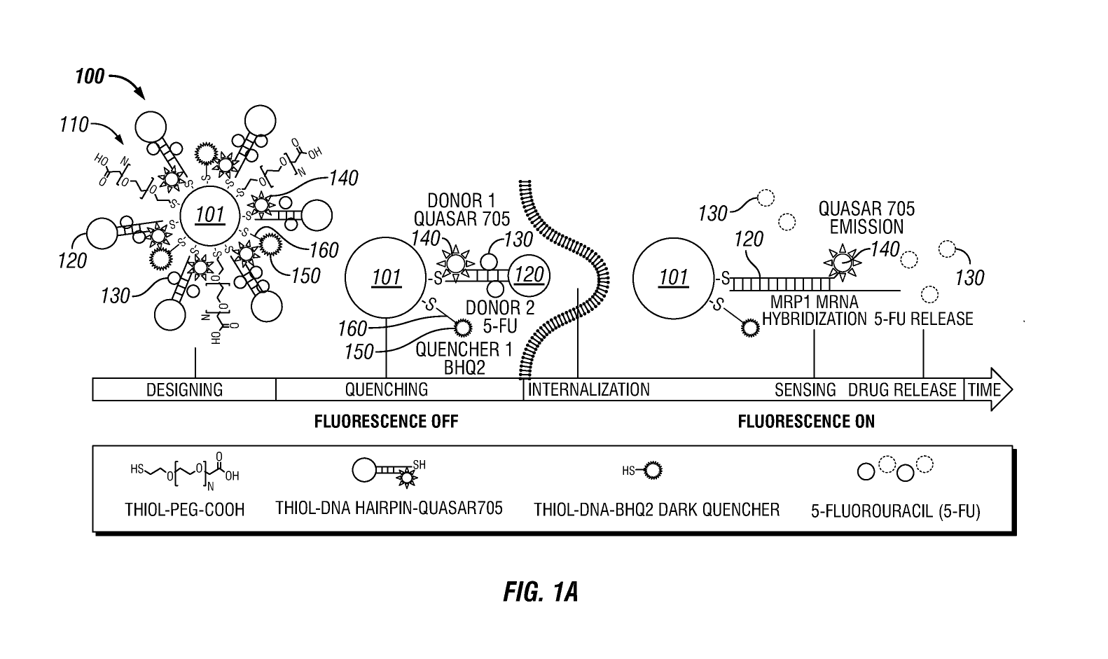

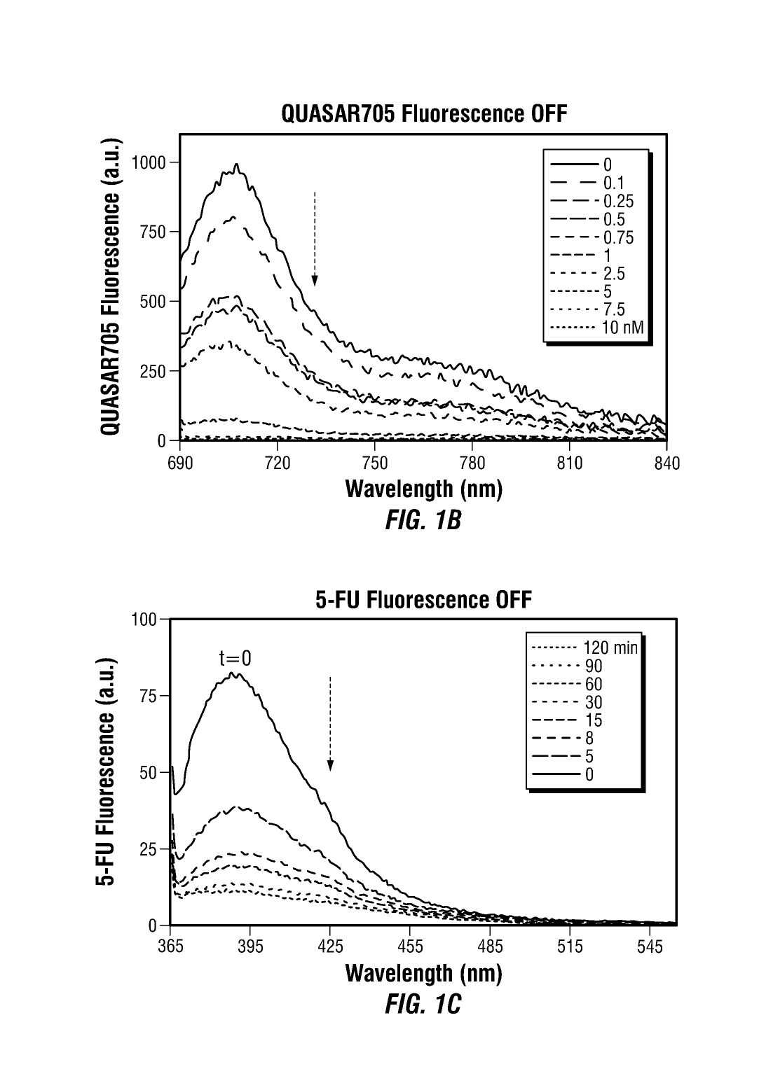

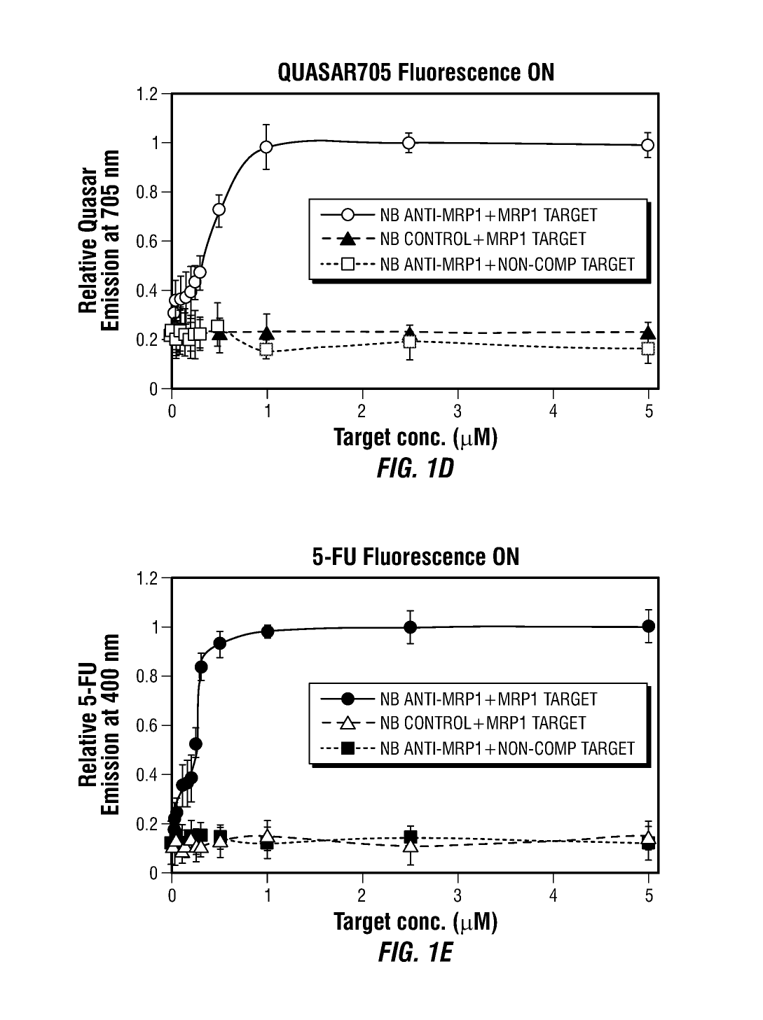

One embodiment of a theranostic nanoprobe is depicted at FIG. 1A. The theranostic nanoprobe 100 of FIG. 1A includes a gold nanoparticle 101 having an average diameter of about 14 nm. The gold nanoparticle 101 is functionalized with [1] thio-PEG-COOH 110 derived from α-Mercapto-ω-carboxy PEG, [2] thio-DNA-hairpin 120 labeled with Quasar® 705 (Q705) 140, which is a near infrared (NIR) dye, and [3] thio-DNA oligonucleotide 160 labeled with Black Hole® Quencher 2 (BHQ2) 150, which is a quencher. The theranostic nanoprobe also includes 5-FU 130 intercalated into the dsDNA of the DNA-hairpins. The thio-DNA-hairpin 120 labeled with Quasar® 705 (Q705) 140 is configured to hybridize to MRP1 mRNA, which is the target molecule.

In the theranostic nanoprobe 100 of FIG. 1A, the gold nanoparticle and the BHQ2 150 function as “quenchers,” while the 5-FU 130 and Q705 140 act as “donors.” Therefore, the fluorescence of the theranostic nanoprobe 100 of FIG. 1A is “off” when the DNA-hairpin 120 labeled with Q705 140 is closed prior to hybridization to a complementary target, because the closed configuration ensures that the distances between [1] BHQ2 150 and Q705 140 and [2] the gold nanoparticle and 5-FU are minimal. The closed configuration also inhibits the release of 5-FU 130 when the fluorescence is “off.”

Conversely, the fluorescence of the theranostic nanoprobe 100 of FIG. 1A is “on” when the DNA-hairpin 120 hybridizes to a complementary target, because the opening the DNA-hairpin 120 increases the distances between [1] the Q705 140 fluorophore and the BHQ2 150 quencher, and [2] the gold nanoparticle and 5-FU, thereby causing an increase in Q705 140 and 5-FU emission. The increase in emission indicates the release of 5-FU 130, which occurs due to the opening of the DNA-hairpin 120. Since release of 5-FU 130 cannot precede hybridization, the theranostic nanoprobe 100 of FIG. 1A is configured to lessen or silence the expression of MRP1, and deliver—at the same time and/or thereafter—the 5-FU 130, in order to circumvent essential MDR mechanisms. In other words, the release of the 5-FU drug is designed to occur only when the DNA-hairpin hybridizes with the complementary mRNA target inside the cell and can be tracked fluorescently once the distance between 5-FU and the gold core increases upon drug emission. Also, the DNA-hairpin labeled with an NIR dye, and the DNA-oligo labeled with a quencher serve as a two pair fluorescence resonance energy transfer/nanoparticle sufficient energy transfer (FRET/NSET) donor quencher nanoconjugate for universal cancer gene therapy and drug delivery. Although the presence of the fluorophore and the quencher-labeled DNA-oligonucleotide permit the theranostic nanoprobe of FIG. 1A to report on the foregoing events, the fluorophore and quencher-labeled DNA-oligonucleotide are not necessary to silence or lessen MDR, achieve drug release, or a combination thereof.

For at least these reasons, the theranostic nanoprobe depicted at FIG. 1A is a bioresponsive hydrogel-nanoprobe that locally senses and inhibits the expression of MRP1, enabling subsequent release of 5-FU drug to resistant triple-negative breast cancer cells (TNBC), both in vitro and in vivo.

### Gold Nanoparticles

In embodiments, the theranostic nanoprobes provided herein comprise gold nanoparticles. The phrase “gold nanoparticle” or “gold nanoparticles” as used herein, refers to a particle or particles comprising gold in at least an amount of 50% by weight, and has an average diameter of less than 100 nm. In one embodiment, the gold nanoparticles of the theranostic nanoprobes provided herein comprise gold in an amount of at least 95% by weight. In another embodiment, the gold nanoparticles of the theranostic nanoprobes provided herein comprise gold in an amount of at least 99% by weight.

The gold nanoparticles of the theranostic nanoprobes provided herein may be selected from those that are commercially available, or made by techniques known in the art, such as the citrate reduction method, e.g., see Lee, P. C. et al., J. PHYS. CHEM. 1982, 86(17), 3391-3395. When the citrate reduction method is used, the gold nanoparticles of the theranostic nanoprobes provided herein may include citrate groups on at least a portion of their surfaces. The citrate groups may be relied upon, at least in part, to functionalize the gold nanoparticles to form the theranostic nanoprobes provided herein. It is well-known, for example, that a thiol functional group can undergo an exchange with a citrate group on the surface of a gold nanoparticle.

In embodiments, the average diameter of the gold nanoparticles of the theranostic nanoprobes provided herein is from about 5 to about 50 nm. In one embodiment, the average diameter of the gold nanoparticles of the theranostic nanoprobes provided herein is from about 5 to about 40 nm. In another embodiment, the average diameter of the gold nanoparticles of the theranostic nanoprobes provided herein is from about 5 to about 30 nm. In a particular embodiment, the average diameter of the gold nanoparticles of the theranostic nanoprobes provided herein is from about 5 to about 20 nm. In a further embodiment, the average diameter of the gold nanoparticles of the theranostic nanoprobes provided herein is from about 8 to about 18 nm. In a still further embodiment, the average diameter of the gold nanoparticles of the theranostic nanoprobes provided herein is from about 10 to about 16 nm. In a certain embodiment, the average diameter of the gold nanoparticles of the theranostic nanoprobes provided herein is about 13 nm. The average diameter of the gold nanoparticles was determined by transmission electron microscopy (TEM) images.

In embodiments, at least two samples of gold nanoparticles having different average diameters may be employed in the theranostic nanoprobes provided herein.

Not wishing to be bound by any particular theory, it is believed that the gold nanoparticles of the theranostic nanoprobes provided herein may act as a second “absorber,” in addition to the quencher used in various embodiments. Therefore, in embodiments, the gold nanoparticles may be used to at least partially quench the emission of at least one chemotherapeutic drug. For example, in embodiments, the emission of the chemotherapeutic agent 5-FU is substantially quenched by the gold nanoparticles of the theranostic nanoprobes provided herein when the gold nanoparticles have an average diameter of about 14 nm. Therefore, the theranostic nanoprobes provided herein may include a dual quencher (gold nanoparticles and quencher used to label an anchor, such as DNA-oligonucleotide) and dual donor (chemotherapeutic drug and DNA-hairpin fluorophore label) system.

Not wishing to be bound by any particular theory, it is believed that the average diameter of the gold nanoparticles may be selected to ensure that the gold nanoparticles at least partially quench the emission of one or more chemotherapeutic drugs. Therefore, in embodiments, gold nanoparticles having a particular average diameter may be used in the theranostic probes provided herein in order to ensure that their region of absorbance at least partially corresponds to the range of emission of one or more chemotherapeutic agents.

### DNA-Hairpin

Generally, the DNA-hairpins used in the theranostic nanoprobes provided herein may include any sequence capable of assuming a “hairpin” configuration, and hybridizing to a target molecule.

As used herein, the phrase “DNA-hairpin” refers to an oligonucleotide capable of assuming a “hairpin” configuration—sometimes referred to as a “hairpin loop” or “stem-loop” configuration—via intramolecular base pairing.

In embodiments, the DNA-hairpin hosts at least one chemotherapeutic agent. The chemotherapeutic agent may be intercalated in the base pairs of the DNA-hairpin.

In embodiments, the theranostic nanoprobes provided herein are functionalized with DNA-hairpin by covalently bonding at least one DNA-hairpin to a gold nanoparticle. The gold nanoparticles, in some embodiments, include citrate groups on their surfaces, and these citrate groups may be contacted with a thiol-DNA-hairpin to bond a thio-DNA-hairpin to the surface of a gold nanoparticle. In one embodiment, DNA-hairpin includes a thio-DNA hairpin.

A gold nanoparticle of the theranostic nanoprobes provided herein may be functionalized with one or more DNA-hairpins having the same sequence. Alternatively, the gold nanoparticles of the theranostic nanoprobes provided herein may be functionalized with two or more DNA-hairpins having different sequences. The sequence or sequences of the DNA-hairpins may be selected to hybridize to at least one desired target molecule.

In embodiments, the gold nanoparticles of the theranostic nanoprobes provided herein are functionalized with DNA-hairpin capable of hybridizing to MRP1 mRNA. In another embodiment, the gold nanoparticles of the theranostic nanoprobes provided herein are functionalized with DNA-hairpin capable of hybridizing to luciferase mRNA. In another embodiment, the theranostic nanoprobes provided herein are functionalized with DNA-hairpin capable of hybridizing to MRP1 mRNA, and DNA-hairpin capable of hybridizing to luciferase mRNA.

In embodiments, the DNA-hairpin employs a mechanism of mRNA knockdown based on antisense DNA technology, which is known in the art (see, e.g., Rakoczy, P. METHODS MOL. MED. 2001, 47, 89-104). This method can inhibit or downregulate the production of MRP1 protein by using antisense DNA oligonucleotides, such as an anti-MRP1 DNA-hairpin, which precisely complements the MRP1 mRNA target sequence present in a cell, including a cancer cell. The anti-MRP1 DNA-hairpin has the ability to interlock or hybridize with the target MRP1 mRNA (blocking specific translation initiation signals of MRP1 gene), thus inhibiting the translation of the target protein.

The DNA-hairpins of the theranostic nanoprobes provided herein also may be labeled with a fluorophore. The fluorophore may be a dye. In embodiments, the dye is a near infrared dye. A “near infrared dye,” as used herein, is a dye that is biocompatible and fluoresces in the near-infrared region. In one embodiment, the near infrared dye is Quasar® 705 (Q705) dye.

The fluorophore label of the DNA-hairpin may be arranged in or on a DNA-hairpin in a position that permits the distance between the quencher and the fluorophore to increase as the DNA-hairpin hybridizes to a target molecule. In embodiments, the fluorophore label is arranged at a position that [1] minimizes the distance between the quencher and the fluorophore prior to the DNA-hairpin's hybridization to a target molecule, [2] maximizes the distance between the quencher and the fluorophore after the DNA-hairpin hybridizes to a target molecule, or [3] a combination thereof.

Generally, the gold nanoparticles of the theranostic nanoprobes provided herein may be functionalized with a number of DNA-hairpins suitable to [1] ensure sufficient hybridization to a target molecule, [2] carry and release a sufficient amount of a chemotherapeutic agent, or [3] a combination thereof.

In embodiments, the ratio of DNA-hairpin:gold nanoparticle is from about 5:1 to about 100:1. In one embodiment, the ratio of DNA-hairpin:gold nanoparticle is from about 5:1 to about 75:1. In another embodiment, the ratio of DNA-hairpin:gold nanoparticle is from about 5:1 to about 50:1. In a particular embodiment, the ratio of DNA-hairpin:gold nanoparticle is from about 10:1 to about 50:1. In a further embodiment, the ratio of DNA-hairpin:gold nanoparticle is from about 20:1 to about 40:1. In a still further embodiment, the ratio of DNA-hairpin:gold nanoparticle is about 30:1.

### Spacer

Generally, the gold nanoparticles of the theranostic nanoprobes provided herein may be functionalized with a spacer. The spacer typically may be any biocompatible molecule, such as a polymer, that does not substantially interfere with the operation of the theranostic nanoprobes provided herein.

The gold nanoparticles of the theranostic nanoprobes provided herein may be functionalized with a spacer prior to functionalization with DNA-hairpin, anchor, or both DNA-hairpin and anchor. Therefore, the spacer may ensure a desired distribution of DNA-hairpin, anchor, or a combination thereof on the surfaces of the gold nanoparticles. The spacer may increase the stability of the theranostic nanoprobes in a biological medium.

The spacer, in embodiments, comprises polyethylene glycol (PEG) and a functional group capable of covalently bonding the spacer to a gold nanoparticle. Such a spacer is referred to herein as a “PEG spacer.” A PEG spacer may include a thiol group that reacts with a citrate group on the surface of a gold nanoparticle in order to bond a thio-PEG spacer to the surface of the gold nanoparticle.

In one embodiment, the PEG spacer includes a terminal carboxylic acid functional group. Not wishing to be bound by any particular theory, it is believed that the charge associated with a carboxylic acid may promote stability due to desirable interactions with the DNA-hairpin and/or DNA-oligonucleotide, when the anchor includes a DNA-oligonucleotide. Therefore, the PEG spacer may include a thio-PEG-COOH spacer, such as an α-Mercapto-ω-carboxy PEG, having an Mₙ of 3500 Da. The spacers may include other functional groups to lend one or more desirable features to the theranostic nanoprobes provided herein.

The gold nanoparticles of the theranostic nanoprobes provided herein may be functionalized with any number of spacer molecules that imparts one or more desired features, such as stability and adequate spacing and distribution of DNA-hairpins and/or anchors, such as DNA-oligonucleotides. In one embodiment, the gold nanoparticles are functionalized with an amount of spacer molecules sufficient to cover from about 5% to about 50% of the surface area of the gold nanoparticles. In another embodiment, the gold nanoparticles are functionalized with an amount of spacer molecules sufficient to cover from about 10% to about 50% of the surface area of the gold nanoparticles. In a further embodiment, the gold nanoparticles are functionalized with an amount of spacer molecules sufficient to cover from about 20% to about 40% of the surface area of the gold nanoparticles. In a particular embodiment, the gold nanoparticles are functionalized with an amount of spacer molecules sufficient to cover about 30% of the surface area of the gold nanoparticles.

### Anchor

The theranostic nanoprobes provided herein may include a gold nanoparticle functionalized with an anchor that is labeled with at least one quencher. In embodiments, the anchor comprises DNA-oligonucleotide, sometimes referred to herein as a “DNA-oligo”. In another embodiment, the anchor includes a biocompatible molecule or polymer that is capable of bonding to the gold nanoparticle. The term “quencher” or phrase “dark quencher,” as used herein, refers to any material that reduces the fluorescence intensity of another material.

Not wishing to be bound by any particular theory, it is believed that associating the at least one quencher with a DNA-oligonucleotide may impart stability to the theranostic nanoprobes provided herein, allow for facile functionalization of the gold nanoparticles with the quencher-labeled DNA-oligonucleotide, or a combination thereof.

The quencher may be selected based on the emission region of the fluorophore associated with the DNA-hairpin. In other words, the quencher may be selected to ensure that the fluorescence of the fluorophore associated with the DNA-hairpin is reduced a desirable amount. In embodiments, the at least one quencher is one that extends to the near-infrared emission wavelengths to overlap with the absorbance range of the at least one fluorophore when the at least one fluorophore is a near-infrared dye. Combinations of different quenchers may be used, and each anchor may be labeled with one or more quenchers having the same or different absorbance ranges.

In embodiments, the gold nanoparticles of the theranostic nanoprobes provided herein are functionalized with DNA-oligonucleotide labeled with Black Hole Quencher® 2 dye (BHQ2) (LGC Biosearch Technologies, CA, USA).

In embodiments, the DNA-oligonucleotide is a thio-DNA-oligonucleotide that is covalently bonded to a gold nanoparticle by contacting the gold nanoparticle with a thiol-DNA-oligonucleotide.

In embodiments, the ratio of anchor:gold nanoparticle is from about 5:1 to about 100:1. In one embodiment, the ratio of anchor:gold nanoparticle is from about 5:1 to about 75:1. In another embodiment, the ratio of anchor:gold nanoparticle is from about 5:1 to about 50:1. In a particular embodiment, the ratio of anchor:gold nanoparticle is from about 10:1 to about 50:1. In a further embodiment, the ratio of anchor:gold nanoparticle is from about 20:1 to about 40:1. In a still further embodiment, the ratio of anchor:gold nanoparticle is about 24:1.

### Chemotherapeutic Agent

Generally, the theranostic nanoprobes provided herein may include any chemotherapeutic agent or drug that is capable of intercalating into a DNA-hairpin. Examples of suitable chemotherapeutic agents include, but are not limited to, indolocarbazoles, such as rebeccamycin; anthracyclines, such as doxorubicin, epirubicin, and mitoxantrone; pyrrolobenzodiazepines, such as tomaymycin and anthramycin; platinum-based agents, such as cisplatin, carboplatin, oxaliplatin, satraplatin, picoplatin, nedaplatin, and triplatin; gemcitabine; vincristine; or any combination thereof, including combinations containing two or more drugs from a single class, and combinations that include one or more agents from different classes.

As used herein, a chemotherapeutic agent that is “capable of intercalating into a DNA-hairpin” is one that is capable of inserting between bases along the dsDNA of a DNA-hairpin. In embodiments, the intercalated chemotherapeutic agent covalently bonds with the DNA-hairpin at one or more sites. The intercalated chemotherapeutic agent may be released when the DNA-hairpin assumes an “open” configuration upon hybridization to a target molecule.

In embodiments, the chemotherapeutic agent forms intrastrand crosslinks, which can prevent polymerase and other DNA binding proteins from functioning properly, which result in DNA synthesis, inhibition of transcription, and induction of mutations.

In embodiments, the theranostic nanoprobes provided herein include a single chemotherapeutic agent. For example, the single chemotherapeutic agent may be 5-FU. In other embodiments, the theranostic nanoprobes provided herein include two or more chemotherapeutic agents. For example, the two or more chemotherapeutic agents may include 5-FU and cisplatin. Other combinations are envisioned.

In one embodiment, the theranostic nanoprobes provided herein include two or more chemotherapeutic agents, and at least one DNA-hairpin of the theranostic nanoprobe is associated with only one of the two or more chemotherapeutic agents. In another embodiment, the theranostic nanoprobes provided herein include two or more chemotherapeutic agents, and at least one DNA-hairpin includes at least one molecule of each of the two or more chemotherapeutic agents.

In embodiments, the ratio of chemotherapeutic agent:gold nanoparticle is from about 10:1 to about 200:1. In other embodiments, the ratio of chemotherapeutic agent:gold nanoparticle is from about 50:1 to about 150:1. In further embodiments, the ratio of chemotherapeutic agent:gold nanoparticle is from about 75:1 to about 125:1. In still further embodiments, the ratio of chemotherapeutic agent:gold nanoparticle is about 100:1.

### Hydrogel

In embodiments, the theranostic nanoprobes provided herein are embedded in a hydrogel. The hydrogel generally may be a biocompatible hydrogel that permits release of the theranostic nanoprobes. The theranostic nanoprobes may be released as the hydrogel degrades. The hydrogel also may allow for the controlled release of the theranostic nanoprobes. The hydrogel may be degradable, injectable, or a combination thereof.

A hydrogel comprising one or more of the theranostic nanoprobes provided herein, in embodiments, may be used on any surface or area. For example, the hydrogels comprising one or more theranostic nanoprobes may be used on or in any internal or external biological tissues, lumens, orifices, or cavities. The biological tissues, lumens, orifices, or cavities may be human or other mammalian tissues, lumens, orifices, or cavities. The biological tissues may be natural or artificially generated. Therefore, the biological tissues may be in vivo or in vitro. The biological tissues may be skin, bone, ocular, muscular, vascular, or an internal organ, such as lung, intestine, heart, liver, etc., or cancerous tissue, including tumors, associated with any biological tissue, including the foregoing.

Not wishing to be bound by any particular theory, it is believed that the local delivery platform using a hydrogel may overcome one or more of the limitations associated with systemic administration, such as low stability, dissociation from vector and short lifetimes, and may avoid or reduce the risk of the uptake of systemically delivered nanoparticles by the liver that makes targeting to other organs difficult.

In embodiments, the hydrogel comprises gold nanobeacons having substantially identical structures. In other embodiments, the hydrogel comprises two or more gold nanobeacons, each having different structures. A “different structure” may be imparted by the use of a different DNA-hairpin, anchor, fluorophore, quencher, size of gold nanoparticle, chemotherapeutic agent, or a combination thereof.

In embodiments, the theranostic nanoprobes are substantially evenly distributed in the hydrogel. In other embodiments, the theranostic nanoprobes are unevenly distributed in the hydrogel. The distribution of theranostic nanoprobes in the hydrogel may be tailored by one of skill in the art in order to obtain a desired release of theranostic nanoprobes from the hydrogel after deployment.

In embodiments, the hydrogels include a formulation comprising a dendrimer component and a polymer component.

In embodiments, the dendrimer component comprises a dendrimer having amines on at least a portion of its surface groups, which are commonly referred to as “terminal groups” or “end groups.” The dendrimer may have amines on from 20% to 100% of its surface groups. In some embodiments, the dendrimer has amines on 100% of its surface groups. In one embodiment, the dendrimer component comprises a dendrimer having amines on less than 75% of its surface groups. In a particular embodiment, the dendrimer component comprises a dendrimer having amines on about 25% of its surface groups.

As used herein, the term “dendrimer” refers to any compound with a polyvalent core covalently bonded to two or more dendritic branches. In some embodiments, the polyvalent core is covalently bonded to three or more dendritic branches. In one embodiment, the amines are primary amines. In another embodiment, the amines are secondary amines. In yet another embodiment, one or more surface groups have at least one primary and at least one secondary amine.

In one embodiment, the dendrimer extends through at least 2 generations. In another embodiment, the dendrimer extends through at least 3 generations. In yet another embodiment, the dendrimer extends through at least 4 generations. In still another embodiment, the dendrimer extends through at least 5 generations. In a further embodiment, the dendrimer extends through at least 6 generations. In still a further embodiment, the dendrimer extends through at least 7 generations.

In one embodiment, the dendrimer has a molecular weight of from about 1,000 to about 1,000,000 Daltons. In a further embodiment, the dendrimer has a molecular weight of from about 3,000 to about 120,000 Daltons. In another embodiment, the dendrimer has a molecular weight of from about 10,000 to about 100,000 Daltons. In yet another embodiment, the dendrimer has a molecular weight of from about 20,000 to about 40,000 Daltons. Unless specified otherwise, the “molecular weight” of the dendrimer refers to the number average molecular weight.

Generally, the dendrimer may be made using any known methods. In one embodiment, the dendrimer is made by oxidizing a starting dendrimer having surface groups comprising at least one hydroxyl group so that at least a portion of the surface groups comprise at least one amine. In another embodiment, the dendrimer is made by oxidizing a starting generation 5 (G5) dendrimer having surface groups comprising at least one hydroxyl group so that at least a portion of the surface groups comprise at least one amine. In yet another embodiment, the dendrimer is made by oxidizing a starting G5 dendrimer having surface groups comprising at least one hydroxyl group so that about 25% of the surface groups comprise at least one amine. In a particular embodiment, the dendrimer is a G5 dendrimer having primary amines on about 25% of the dendrimer's surface groups.

In one embodiment, the dendrimer is a poly(amidoamine)-derived (PAMAM) dendrimer. In another embodiment, the dendrimer is a G5 PAMAM-derived dendrimer. In yet another embodiment, the dendrimer is a G5 PAMAM-derived dendrimer having primary amines on about 25% of the dendrimer's surface groups.

In one embodiment, the dendrimer is a poly(propyleneimine)-derived dendrimer.

In certain embodiments, the dendrimer component is combined with a liquid to form a dendrimer component solution. In one embodiment, the dendrimer component solution is an aqueous solution. In one embodiment, the solution comprises water, phosphate buffer saline (PBS), Dulbecco's Modified Eagle's Medium (DMEM), or any combination thereof. In one embodiment, the dendrimer component concentration in the dendrimer component solution is about 5% to about 25% by weight. In another embodiment, the dendrimer component concentration in the dendrimer component solution is about 10% to about 20% by weight. In a further embodiment, the dendrimer component concentration in the dendrimer component solution is about 11% to about 15% by weight.

In some instances, the dendrimer component or dendrimer component solution further includes one or more additives. Generally, the amount of additive may vary depending on the application, tissue type, concentration of the dendrimer component solution, the type of dendrimer component, concentration of the polymer component solutions, and/or the type of polymer component. Example of suitable additives, include but are not limited to, pH modifiers, thickeners, antimicrobial agents, colorants, surfactants, and radio-opaque compounds. Specific examples of these types of additives are described herein. In one embodiment, the dendrimer component solution comprises a foaming additive.

In particular embodiments, the dendrimer component or dendrimer component solution includes one or more drugs. In such embodiments, the hydrogel may serve as a matrix material for controlled release of the one or more drugs. The drug may be essentially any drug suitable for local, regional, or systemic administration from a quantity of the hydrogel that has been applied to one or more tissue sites in a patient. In one embodiment, the drug comprises a thrombogenic agent. Non-limiting examples of thrombogenic agents include thrombin, fibrinogen, homocysteine, estramustine, and combinations thereof. In another embodiment, the drug comprises an anti-inflammatory agent. Non-limiting examples of anti-inflammatory agents include indomethacin, salicyclic acid acetate, ibuprophen, sulindac, piroxicam, naproxen, and combinations thereof. In still another embodiment, the drug comprises an anti-neoplastic agent. In still other embodiments, the drug is one for gene therapy. For example, the drug may comprise siRNA molecules to combat cancer. Other drugs are envisioned.

In other particular embodiments, the dendrimer component or dendrimer component solution includes one or more cells. Alternatively or in addition, the polymer component or polymer component solution includes one or more cells. For example, in any of these embodiments, the hydrogels may serve as a matrix material for delivering cells to a tissue site at which the hydrogel has been applied. In embodiments, the cells may comprise endothelial cells (EC), endothelial progenitor cells (EPC), hematopoietic stem cells, or other stem cells. In one embodiment, the cells are capable of releasing factors to treat cardiovascular disease and/or to reduce restenosis. Other types of cells are envisioned.

Generally, the polymer component includes a polymer and/or oligomer with one or more functional groups capable of reacting with one or more functional groups on a biological tissue and/or one or more functional groups on the dendrimer component.

In certain embodiments, the polymer is at least one polysaccharide. In these embodiments, the at least one polysaccharide may be linear, branched, or have both linear and branched sections within its structure. Generally, the at least one polysaccharide may be natural, synthetic, or modified—for example, by cross-linking, altering the polysaccharide's substituents, or both. In one embodiment, the at least one polysaccharide is plant-based. In another embodiment, the at least one polysaccharide is animal-based. In yet another embodiment, the at least one polysaccharide is a combination of plant-based and animal-based polysaccharides. Non-limiting examples of polysaccharides include, but are not limited to, dextran, chitin, starch, agar, cellulose, hyaluronic acid, or a combination thereof.

In certain embodiments, the at least one polymer has a molecular weight of from about 1,000 to about 1,000,000 Daltons. In one embodiment, the at least one polymer has a molecular weight of from about 5,000 to about 15,000 Daltons. Unless specified otherwise, the “molecular weight” of the polymer refers to the number average molecular weight.

In some embodiments, the polymer is functionalized so that its structure includes one or more functional groups that will react with one or more functional groups on a biological tissue and/or one or more functional groups on the dendrimer component. In other embodiments, the polymer is functionalized so that its structure includes three or more functional groups that will react with one or more functional groups on a biological tissue and/or one or more functional groups on the dendrimer component. In one embodiment, the functional groups incorporated into the polymer's structure is aldehyde.

In certain embodiments, the polymer's degree of functionalization is adjustable. The “degree of functionalization” generally refers to the number or percentage of groups on the polymer that are replaced or converted to the desired one or more functional groups. The one or more functional groups, in particular embodiments, include aldehydes, substituents capable of photoreversible dimerization, or a combination thereof. In one embodiment, the degree of functionalization is adjusted based on the type of tissue to which the hydrogel is applied, the concentration(s) of the components, and/or the type of polymer or dendrimer used in the hydrogel. In one embodiment, the degree of functionalization is from about 10% to about 75%. In another embodiment, the degree of functionalization is from about 15% to about 50%. In yet another embodiment, the degree of functionalization is from about 20% to about 30%.

In one embodiment, the polymer is a polysaccharide having from about 10% to about 75% of its hydroxyl groups converted to aldehydes. In another embodiment, the polymer is a polysaccharide having from about 20% to about 50% of its hydroxyl groups converted to aldehydes. In yet another embodiment, the polymer is a polysaccharide having from about 10% to about 30% of its hydroxyl groups converted to aldehydes.

In one embodiment, the polymer is dextran with a molecular weight of about 10 kDa. In another embodiment, the polymer is dextran having about 50% of its hydroxyl group converted to aldehydes. In a further embodiment, the polymer is dextran with a molecular weight of about 10 kDa and about 50% of its hydroxyl groups converted to aldehydes.

In some embodiments, a polysaccharide is oxidized to include a desired percentage of one or more aldehyde functional groups. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization.

In certain embodiments, the polymer component is combined with a liquid to form a polymer component solution. In one embodiment, the polymer component solution is an aqueous solution. In one embodiment, the solution comprises water, PBS, DMEM, or any combination thereof.

Generally, the polymer component solution may have any suitable concentration of polymer component. In one embodiment, the polymer component concentration in the polymer component solution is about 5% to about 40% by weight. In another embodiment, the polymer component concentration in the polymer component solution is about 5% to about 30% by weight. In yet another embodiment, the polymer component concentration in the polymer component solution is about 5% to about 25% by weight. Typically, the concentration may be tailored and/or adjusted based on the particular application, tissue type, and/or the type and concentration of dendrimer component used.

The polymer component or polymer component solution may also include one or more additives. In one embodiment, the additive is compatible with the polymer component. In another embodiment, the additive does not contain primary or secondary amines. Generally, the amount of additive varies depending on the application, tissue type, concentration of the polymer component solution, the type of polymer component and/or dendrimer component. Examples of suitable additives, include, but are not limited to, pH modifiers, thickeners, antimicrobial agents, colorants, surfactants, radio-opaque compounds, and the other additives described herein. In other embodiments, the polymer component solution comprises a foaming agent.

In certain embodiments, the pH modifier is an acidic compound. Examples of acidic pH modifiers include, but are not limited to, carboxylic acids, inorganic acids, and sulfonic acids. In other embodiments, the pH modifier is a basic compound. Examples of basic pH modifiers include, but are not limited to, hydroxides, alkoxides, nitrogen-containing compounds other than primary and secondary amines, basic carbonates, and basic phosphates.

Generally, the thickener may be selected from any known viscosity-modifying compounds, including, but not limited to, polysaccharides and derivatives thereof, such as starch or hydroxyethyl cellulose.

Generally, the surfactant may be any compound that lowers the surface tension of water. In one embodiment, the surfactant is an ionic surfactant—for example, sodium lauryl sulfate. In another embodiment, the surfactant is a neutral surfactant. Examples of neutral surfactants include, but are not limited to, polyoxyethylene ethers, polyoxyethylene esters, and polyoxyethylene sorbitan.

In particular embodiments, the polymer component or polymer component solution includes one or more drugs. In such embodiments, the hydrogel may serve as a matrix material for controlled release of drug. The drug may be essentially any drug suitable for local, regional, or systemic administration from a quantity of the hydrogel that has been applied to one or more tissue sites in a patient. In one embodiment, the drug comprises a thrombogenic agent. Non-limiting examples of thrombogenic agents include thrombin, fibrinogen, homocysteine, estramustine, and combinations thereof. In another embodiment, the drug comprises an anti-inflammatory agent. Non-limiting examples of anti-inflammatory agents include indomethacin, salicyclic acid acetate, ibuprophen, sulindac, piroxicam, naproxen, and combinations thereof. In still another embodiment, the drug comprises an anti-neoplastic agent. In still other embodiments, the drug is one for gene or cell therapy. For example, the drug may comprise siRNA molecules to combat cancer. Other drugs are envisioned.

In other particular embodiments, the polymer component or polymer component solution includes one or more cells. For example, the hydrogel may serve as a matrix material for delivering cells to a tissue site at which the hydrogel has been applied. In embodiments, the cells may comprise endothelial cells (EC), endothelial progenitor cells (EPC), hematopoietic stem cells, or other stem cells. In one embodiment, the cells are capable of releasing factors to treat cardiovascular disease and/or to reduce restenosis. Other types of cells are envisioned.

Generally, the hydrogels described herein may be formed by combining the polymer component or polymer component solution, and the dendrimer component or dendrimer component solution in any manner. In some embodiments, the polymer component or polymer component solution, and the dendrimer component or dendrimer component solution are combined before contacting a biological tissue with the hydrogel. In other embodiments, the polymer component or polymer component solution, and the dendrimer component or dendrimer component solution are combined, in any order, on a biological tissue.

In embodiments, one or more theranostic nanoprobes are added to the hydrogel after hydrogel formation. In other embodiments, one or more theranostic nanoprobes are added to at least one component or component solution of the hydrogel prior to hydrogel formation. For example, one or more theranostic nanoprobes may be added to a polymer component or polymer component solution before the polymer component or polymer component solution is combined with a dendrimer component or dendrimer component solution to form a hydrogel. Conversely, the one or more theranostic nanoprobes may be added only to the dendrimer component or dendrimer component solution prior to hydrogel formation, or to both the polymer component or polymer component solution and the dendrimer component or dendrimer component solution.

### Methods for Treating Biological Tissue

Methods for treating biological tissue, including cancerous tissue, such as tumors, are provided. In embodiments, a biological tissue is treated by providing a hydrogel comprising an embedded theranostic nanoprobe, and disposing, i.e., applying, the hydrogel on a surface of a biological tissue. The surface of the biological tissue may be the surface of the biological tissue in need of treatment. Alternatively, the surface of the biological tissue may be the surface of a biological tissue not in need of treatment, but proximate to a biological tissue in need of treatment. In other words, the hydrogel may be disposed at any location that permits the theranostic nanoprobes, upon release or otherwise, to contact a biological tissue in need of treatment. The hydrogel may be provided in any shape, such as a sphere, disk, film, or the like.

As used herein, the term “treating” generally refers to improving the response of at least one biological tissue to which one or more theranostic nanoprobes is applied. In some embodiments, the “response” that is improved or enhanced includes a reduction in tumor size, reducing the MDR of cancerous cells, or a combination thereof. The theranostic nanoprobes provided herein may be employed after cancer cells have acquired resistance to a specific chemotherapeutic drug. The theranostic nanoprobes provided herein also may be used as prophylaxis, including prior to the establishment of at least some drug resistance.

In embodiments, a biological tissue is treated by combining two components to form a hydrogel, wherein at least one of the components comprises a theranostic nanoprobe. In one embodiment, the hydrogel is formed by combining [1] a first solution comprising a polymer component, wherein the polymer component comprises a polymer having three or more aldehyde groups, and [2] a second solution comprising a dendrimer component, wherein the dendrimer component comprises a dendrimer having at least 2 branches with one or more surface groups. The first solution, the second solution, or both the first solution and the second solution may include a theranostic nanoprobe. The first and second solutions may be combined by any means known in the art, including a double-barreled syringe fitted with a mixing tip. The syringe may be used to apply the components to the surface of a biological tissue, inject the components into a biological tissue, or a combination thereof.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

Throughout this application, and in the following examples, differences between groups were examined using Student's paired t test through SPSS statistical package (version 17, SPSS, Inc., IL, USA). All error bars used in this report are mean±SD of at least 3 independent experiments. Statistically significant P values were indicated in figures and/or legends as ***, P<0.005; **, P<0.02; *, P<0.05. All in vivo experiments used 5 mice per treatment group unless otherwise noted.

### Example 1

### Synthesis of Bare-Gold Nanoparticles

Gold nanoparticles were synthesized by a citrate reduction method, which is well-known in the art, e.g., see Lee, P. C. et al., J. PHYS. CHEM. 1982, 86(17), 3391-3395.

The gold nanoparticles produced by the methods of this example had an average diameter of 13.8±3.4 nm.

In the first step, 225 mL of 1 mM hydrogen tetrachloroaureate (III) hydrate (Sigma-Aldrich, USA)(88.61 mg) was combined with 500 mL of distilled water, heated, and stirred under reflux. When boiling began, 25 mL of 38.8 mM sodium citrate dihydrate (Sigma-Aldrich, USA)(285 mg) was added, which resulted in a red solution. The solution was kept under ebullition with vigorous stirring, and protected from light for 30 minutes. The solution was then cooled down, and still protected from light.

The resulting bare-gold nanoparticles were characterized by Transmission Electron Microscopy (TEM) and UV-Vis molecular absorption spectra.

### Example 2

### Synthesis of PEGylated-Gold Nanoparticles

PEGylated-gold nanoparticles were produced in this example with commercial hetero-functional PEG, specifically a α-Mercapto-ω-carboxy PEG solution (HS—C₂H₄—CONH-PEG-O—C₃H₆—COOH)(3500 Da)(Sigma-Aldrich, USA)(see, e.g., Sanz, V., et al., JOURNAL OF NANOPARTICLE RESEARCH, 2012, 14, and Conde, J. et al. ACS NANO, 2012, 6, 8316-8324). The PEGylated-gold nanoparticles produced by this example were functionalized with a 30% saturated surface of the α-Mercapto-ω-carboxy PEG.

Not wishing to be bound by any particular theory, it is believed that the 30% of saturated PEG layer allowed the incorporation of additional thiolated components, such as the thiolated DNA-hairpin-Quasar 705 nm, and the thiolated-oligo-BHQ2 quencher.

In this example, 10 nM of the bare-gold nanoparticles of Example 1 were dispersed in an aqueous solution of 0.01×PBS (Cytodiagnostics, Ontario, Canada), and then combined with 0.0006 mg/mL of the commercial hetero-functional PEG solution (α-Mercapto-ω-carboxy PEG solution (HS—C₂H₄—CONH-PEG-O—C₃H₆—COOH)(3500 Da)) in an aqueous solution of SDS (0.028%).

The mixture was then incubated for 16 hours at room temperature. Excess PEG was removed by centrifugation (15,000×rpm, 30 min. 4° C.), and quantified by a modification of the Ellman's Assay (see, e.g., Conde, J. et al. ACS NANO, 2012, 6, 8316-8324, and Conde J. et al, BIOMATERIALS, 2013, 24, 2516-2523).

The excess of thiolated chains in the supernatant was quantified by interpolating a calibration curve set by reacting 200 μL of α-Mercapto-ω-carboxy PEG solution in 100 μL of phosphate buffer (0.5 M, pH 7) with 7 μL 5,5′-dithio-bis(2-nitrobenzoic)acid (DTNB, 5 mg/mL) in phosphate buffer (0.5 M, pH 7) and measuring the absorbance at 412 nm after 10 minutes of reaction.

The linear range for the PEG chain obtained by this method was 0-0.1 mg/mL (Abs at 412 nm=6.8991×[PEG, mg/mL]+0.0588). The number of exchanged chains was calculated by the difference between the amount determined by this assay and the initial amount incubated with the gold nanoparticles.

There was a point at which the nanoparticles became saturated with the thiolated layer, and were not able to take up more thiolated chains, which indicated maximum coverage per gold nanoparticle, i.e., 0.02 mg/mL of PEG.

The gold nanoparticles were functionalized with 0.006 mg/mL of PEG corresponding to 30% of PEG saturation on the nanoparticle's surface. The stability of gold nanoparticles with increasing PEG concentration was evaluated by measuring the ratio of absorbance 520/600 nm, and measuring the ratio between non-aggregated and aggregated nanoparticles, as known in the art. A maximum stability was achieved upon functionalization with 0.02 mg/mL of PEG, which validated the results regarding PEG saturation.

### Example 3

### Synthesis of Dark-Gold Nanobeacons

Three different sequences of gold nanobeacons were prepared by the methods of this example: (1) a nanobeacon anti-MRP1, which detected and inhibited MRP1 mRNA, (2) a nanobeacon anti-Luc, which hybridized with luciferase mRNA and released a drug, however, did not target MRP1, and (3) a nanobeacon nonsense, which was designed not to hybridize with any target within the genome).

Therefore, in addition to designing an anti-MRP1 nanobeacon that detected and inhibited MRP1 mRNA, anti-Luc nanobeacons (which hybridized with luciferase mRNA and released the drug without targeting MRP1), and nonsense nanobeacons (which did not hybridize with any target) were developed as controls for this example.

The thiol-DNA-hairpin Quasar® 705 sequences are shown in the following table:

Oligomers sequences used in dark-gold nanobeacons
assembly.

| Oligomers | Sequence and modifications |
|---|---|
| nanobeacon | Thiol- 5′tttgcatGGCTACATTCAGATGACAC |
| anti-MRP1 | atgcaaa 3′ -Q705 |
| nanobeacon | Thiol- 5′ tttgcatCGTACGCGGAATACTTCGA |
| anti-Luc | atgcaaa 3′ -Q705 |
| nanobeacon | Thiol- 5′ tttgcatTTCTCCGAACGTGTCACGT |
| nonsense | atgcaaa 3′-Q705 |
| Oligo-BHQ2 | Thiol- 5′ TTTGGG 3′ -BHQ2 |
| dark quencher | |
| MRP1 target | 5′-GTGTCATCTGAATGTAGCC-3′ |

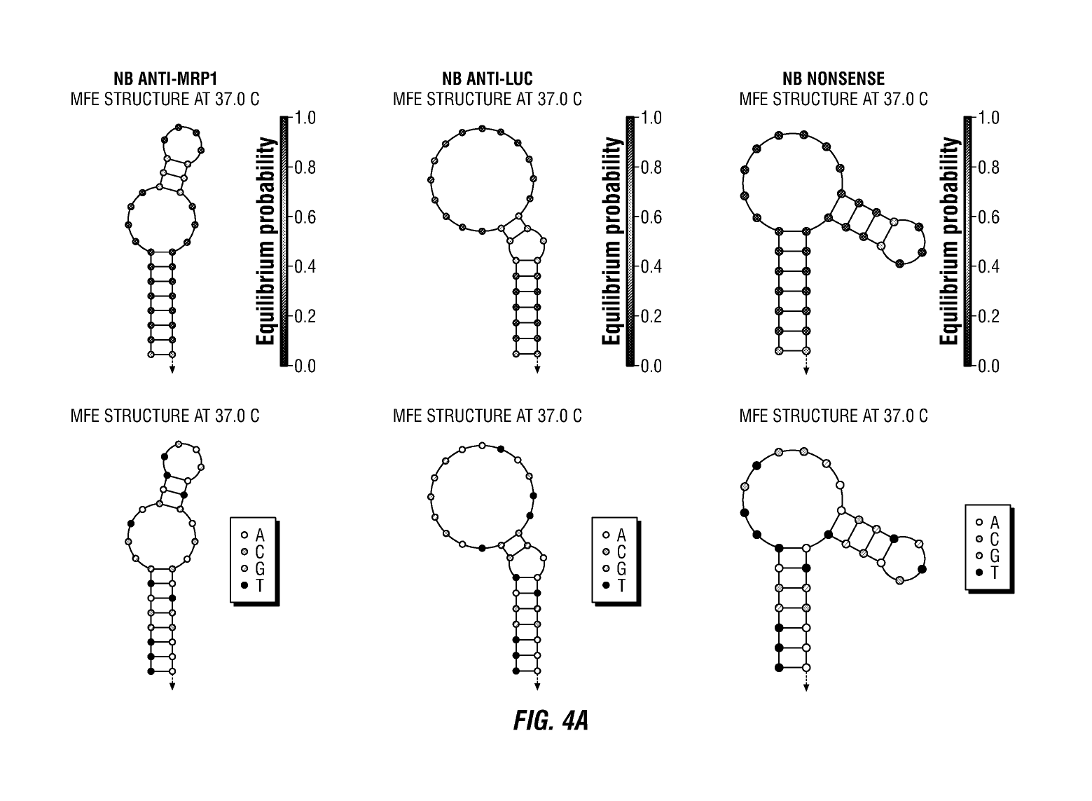

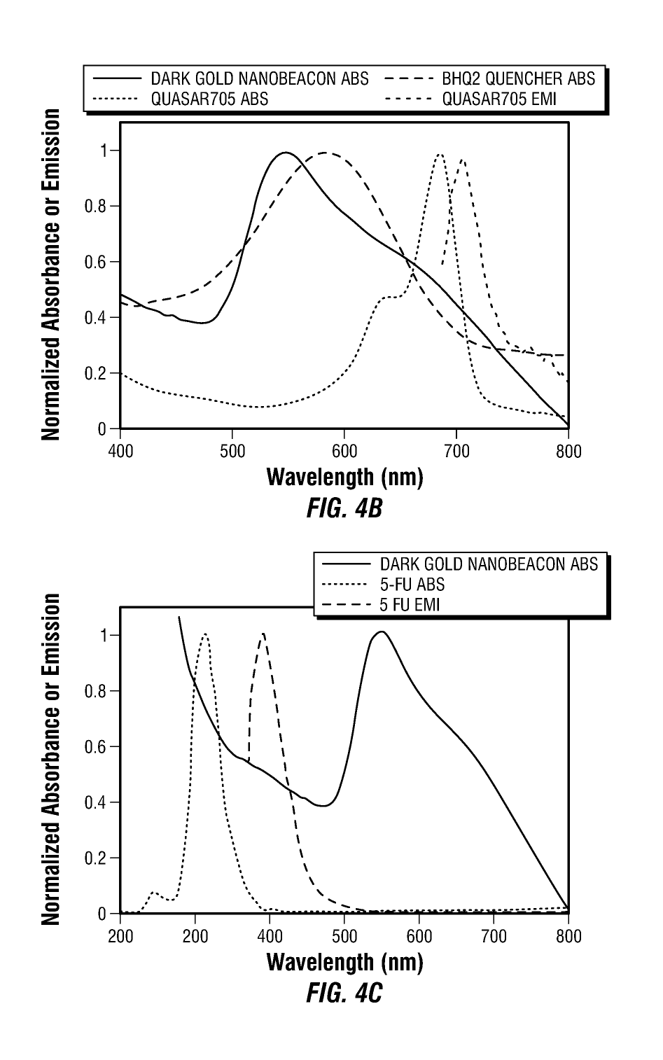

FIG. 4A depicts two-dimensional structures of the sequences for the anti-MRP1, anti-Luc, and nonsense nanobeacons at 37° C., as predicted by NUPACK (Caltech, USA).

The nanobeacons of this example were also functionalized with a DNA-oligo-BHQ2 dark quencher. This was achieved by suspending the thiolated nucleotides (Sigma-Aldrich, USA)—thiol-DNA-hairpin Quasar®705 and DNA-oligo-BHQ2 dark quencher—in 1 mL of 0.1 M dithiothreitol (DTT)(Sigma-Aldrich, USA), followed by three extractions with ethyl acetate, and further purification through a desalting NAP-5 column (GE Healthcare, USA) using 10 mM phosphate buffer (pH 8) as eluent.

Following oligonucleotide quantification via UV/Vis spectroscopy, each oligomer was added to the solution of PEGylated gold nanoparticles of Example 2 in a 50:1 ratio.

AGE I solution (2% (w/v) SDS, 10 mM phosphate buffer (pH 8)) was added to the mixture to achieve a final concentration of 10 mM phosphate buffer (pH 8), 0.01% (w/v) SDS. The solution was sonicated for 10 seconds using an ultrasound bath and incubated at room temperature for 20 minutes. Afterwards, the ionic strength of the solution was increased sequentially in 50 mM NaCl increments by adding the required volume of AGE II solution (1.5 M NaCl, 0.01% (w/v) SDS, 10 mM phosphate buffer (pH 8)) up to a final concentration of 10 mM phosphate buffer (pH 8), 0.3 M NaCl, 0.01% (w/v) SDS.

After each increment, the solution was sonicated for 10 seconds and incubated at room temperature for 20 minutes before the next increment. Following the last addition, the solution was left to rest for an additional 16 hours at room temperature. Then, the functionalized dark-gold nanobeacons were centrifuged for 20 minutes at 15,000 rpm, the oil precipitate washed three times with MilliQ water, and redispersed in MilliQ water. The resulting dark-gold nanobeacons were stored in the dark at 4° C. until further use. Characterization of the dark-gold nanobeacons was performed by Dynamic Light Scattering (DLS) with a Wyatt Dyna Pro Plate Reader, UV/Vis spectroscopy, and TEM.

The average number of labeled beacons per nanoparticle was assessed by the quantification of the excess of the thiolated oligonucleotides (beacons) from the gold nanobeacon synthesis.

All of the supernatants containing the unbound oligonucleotides were measured by monitoring the emission spectra of Quasar® 705 (Exc=675 nm) dye in a microplate reader (Varioskan Flash Multimode Reader, Thermo Scientific). All the gold nanoparticles samples and the standard solutions of the thiol-oligonucleotide beacon were kept at the same pH and ionic strength and calibration for all measurements.

Fluorescence emission was converted to molar concentrations of the thiol modified oligonucleotide by interpolation from a standard linear calibration curve. Standard curves were prepared with known concentrations of beacon using the same buffer pH and salt concentrations. The average number of molecular beacon strands per particle was obtained by dividing the oligonucleotide molar concentration by the gold nanoparticle concentration.

The total number of thiolated-beacon chains that could be attached to each gold nanoparticle is shown in the following table:

Physico-chemical properties of Dark-gold nanobeacons.

| | | | Hairpin- | Oligo-BHQ2 | 5-FU |
|---|---|---|---|---|---|
| | SPR | Size | Q705 mol | mol per | mol per |
| Dark-gold nanbeacons | peak | (nm)* | per particle | particle | particle |
| nanobeacon anti-MRP1 | 548 | 25.6 ± 1.4 | 28.6 ± 5.1 | 27.4 ± 4.8 | NA |
| nanobeacon anti-Luc | 548 | 25.1 ± 1.3 | 29.4 ± 3.7 | 27.7 ± 1.9 | NA |
| nanobeacon nonsense | 548 | 25.8 ± 0.8 | 28.1 ± 3.9 | 26.9 ± 4.5 | NA |
| nanobeacon anti-MRP1 + 5-FU | 550 | 30.9 ± 0.5 | 28.6 ± 5.1 | 23.4 ± 4.8 | 98.4 ± 6.7 |
| nanobeacon anti-Luc + 5-FU | 550 | 31.2 ± 0.7 | 29.4 ± 3.7 | 22.7 ± 1.9 | 101.2 ± 8.6 |
| nanobeacon nonsense + 5-FU | 550 | 31.0 ± 0.4 | 28.1 ± 3.9 | 23.9 ± 4.5 | 99.3 ± 8.2 |

*Determined by Dynamic Light Scattering (DLS).

Transmission electron microscopy (TEM) images of the gold nanobeacons loaded with 5-FU of this particular example showed an average diameter of the gold nanoparticle of 13.8±3.4 nm. The Q705-DNA-hairpin:gold nanoparticle ratio was about 30:1 in this example. The BHQ2-DNA-oligonucleotide:gold nanoparticle ratio was about 24:1 in this example. The ratio of 5-FU:gold nanoparticle was about 100:1.

The mean particle diameter of the gold nanobeacons of this particular example was about 25.5 (±1.2) nm without 5-FU, and about 31.1 (±0.5) nm loaded with 5-FU, with a surface plasmon resonance (SPR) peak around 548-550 nm (see FIG. 1D), as measured by DLS and UV-Vis extinction profiles, respectively.

The choice of fluorophores and quenchers of this particular example permitted [1] the gold nanobeacon including BHQ2 dark quencher (which extends to the near-infrared emission wavelengths) to overlap with the absorbance range of the Q705 fluorophore, and [2] the SPR profile of the gold nanobeacon to overlay the absorbance wavelength from the 5-FU at 450 nm.

These correlations are depicted at FIG. 4B and FIG. 4C. FIG. 4B depicts the normalized absorbance and emission spectra of the gold nanoparticles functionalized with a DNA oligonucleotide that includes the BHQ2 dark quencher. FIG. 4C depicts the normalized absorbance and emission spectra of gold nanoparticles functionalized with a DNA oligonucleotide labeled with Q705 and free drug 5-FU.

In this particular example, the BHQ2 groups functioned as a quencher for the Q705 (donor 1) and the gold nanoparticles functioned as a quencher for the 5-FU (donor 2). The NIR dye Q705 and the quencher BHQ2 (having a strong absorption from 599 nm to 670 nm) were chosen for this example to permit efficient quenching in the nanobeacon basal state with restored fluorescence upon conjugation to the appropriate target.

Not wishing to be bound by any particular theory, it was believed that the fluorescent emission from the 5-FU (emission=450 nm) was efficiently quenched by the ˜14 nm gold nanoparticles due to the nanosurface energy transfer (NSET) effect. It was believed that this effect, at least in part, rendered the gold nanobeacons of this example the base of the fluorescent theranostic nanoprobe of this example, because drug release could be monitored by monitoring changes in fluorescence.

It was observed that the fluorescence emission intensity of the Q705-DNA-hairpins decreased after the reaction with the nanobeacons functionalized with BHQ2-DNA-oligonucleotides. It was believed that the binding between the Q705-DNA-hairpins and the gold nanobeacons occurred by direct quasi-covalent bond between the thiol group on the 5′ end of the oligonucleotide and the gold surface, by ligand-exchange between the citrate groups on the surface of the gold nanoparticles and the thiol groups.

As shown at FIG. 1B, when the concentration of Q705-DNA-hairpins was fixed at 0.5 μM, the increase in the concentration of gold nanoparticles functionalized with BHQ2-DNA-oligonucleotides from 0 to 10 nM in the reaction mixture resulted in a significant increase in Q705 fluorescence quenching. This was believed to demonstrate the presence of nanoparticle sufficient energy transfer (NSET) between the Q705 groups and the gold nanoparticles functionalized with BHQ2-DNA-oligonucleotides. These experimental data demonstrated a quenching efficiency of nearly 99% when the concentration of gold nanoparticles functionalized with BHQ2-DNA-oligonucleotides reached 1 nM, and nearly 100% when the concentration of gold nanoparticles functionalized with BHQ2-DNA-oligonucleotide reached 2.5 nM. Therefore, the BHQ2-DNA-oligonucleotide provided powerful fluorescence quenching for Q705 emission in this example.

The same phenomenon occurred for 5-FU loading on the gold nanoparticles functionalized with BHQ2-DNA-oligonucleotides, as depicted at FIG. 1C. When 1 mM of 5-FU was loaded on 2.5 mM of gold nanoparticles functionalized with BHQ2-oligonucleotides, the fluorescence emission spectra of 5-FU after incubation in phosphate buffer (pH 7.0, 10 mM) for different periods of time (0 to 120 minutes) confirmed a significant increase in 5-FU fluorescence quenching (FIG. 1C). This phenomenon was believed to confirm the formation of BHQ2-functionalized gold nanoparticles loaded with 5-FU.

Conversely, the Q705 emission was restored after hybridization with the complementary ss DNA target. Increasing amounts of complementary target resulted in [1] elevated emission from the Q705 dye until it reached a plateau at about 1 μM of target (see FIG. 1D) and [2] 5-FU release, with nearly complete drug release at 1 μM of target (see FIG. 1E).

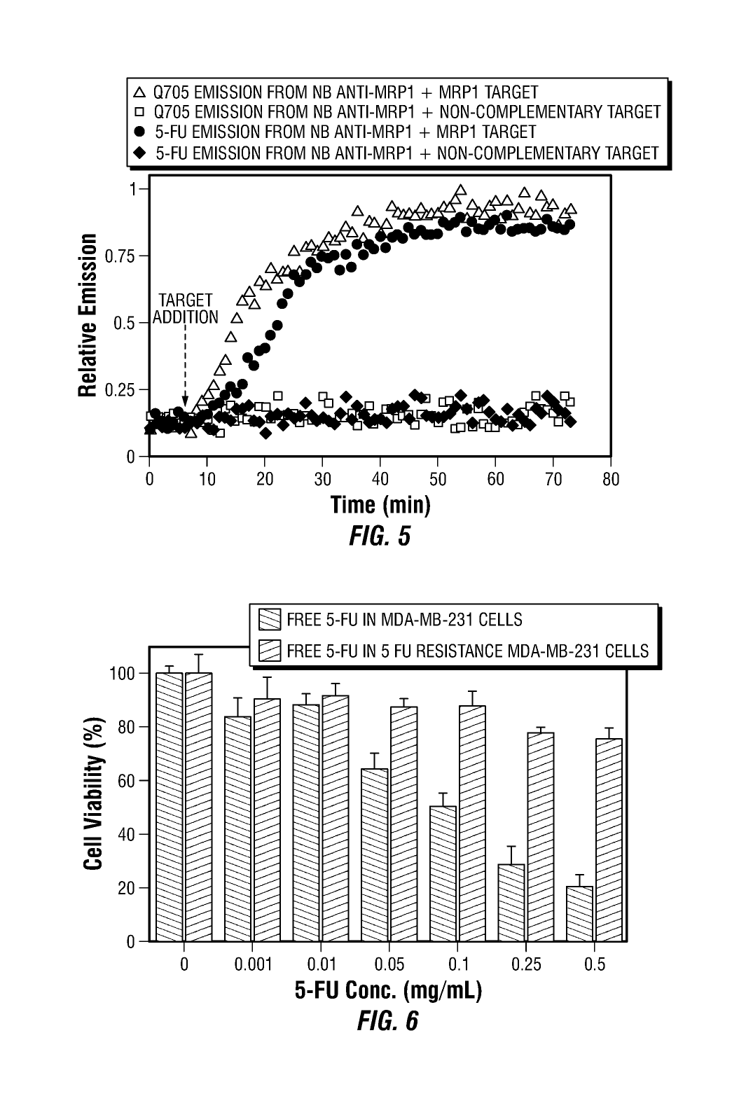

The release kinetics of the intercalated drug following fluorescence of the 5-FU were measured for 75 minutes following addition of 1 μM of target, and compared to the fluorescence of the Q705 dye, as depicted at FIG. 5. The kinetics of the 5-FU release followed the “opening” of the DNA-hairpins subsequent to the hybridization of a complementary target.

For the detection of specific targets, 2.5 nM of nanobeacon anti-MRP1 and nanobeacon nonsense in 10 mM of phosphate buffer (pH 7) was added to 5 nM of complementary MRP1 target (Sigma-Aldrich, USA).

All measurements of this example were performed in a microplate reader (Varioskan Flash Multimode Reader, Thermo Scientific, MA, USA) programmed to incubate the reactions for 120 minutes at 37° C. while recording the fluorescence intensity every 2 minutes at an excitation wavelength of 675 nm for Quasar705-labeled gold nanobeacon, and at an excitation of 350 nm for 5-FU.

### Example 4

### Stability and Specificity Assays for Dark-Gold Nanobeacons

To evaluate whether the reductive cell environment would cause detachment of the thiol-DNA-hairpin from the dark-gold nanobeacons, the resistance to glutathione (GST) was evaluated. To mimic the behavior of the intra-cellular milieu, 2.5 nM of the nanobeacon anti-MRP1 was incubated with 0.1, 1, 5 (physiological concentration), 10, and 100 mM of GST (Sigma-Aldrich, USA) at 37° C. for up to 120 minutes, while measuring fluorescence intensity every 2 minutes (Exc/Emi=675/705 nm for Quasar705-labeled gold nanobeacon; Exc/Emi=350/400 nm for 5-FU). The same procedure was performed for stability at different pHs (4.5, 5.5, 6.5, 7, 7.5, and 8), different temperatures (25° C., 37° C., 42° C., and 45° C.), and different DNase concentrations (0.5, 1, 2.5, and 5 Units).

The stability of dark-gold nanobeacons loaded with 5-FU towards variations with temperature, pH, DNase concentration, and Glutathione (GSH) concentration also was evaluated. The data confirmed the stability of the dark-gold nanobeacons loaded with 5-FU at room and physiological (37° C.) temperatures, and over a wide pH range (4.8 to 8), as well as to intracellular concentrations of GSH and DNase. Specifically, the effect of temperature on nanobeacon signal and release of 5-FU was tested [1] at temperatures of 25, 37, 42, and 45° C. over time, [2] at pH of 4.5, 5.5, 6.5, 7, 7.5, and 8 at 37° C., [3] at increasing concentrations of glutathione (0.1, 1, 5 (physiological concentration), 10, and 100 mM) over time at 37° C., and [4] at increasing concentrations of DNase I (0.5, 1, 2.5, and 5 Units) over time at 37° C.

### Example 5

### In Vitro Dark-Gold Nanobeacon's Delivery

To test the ability of dark gold nanobeacons loaded with 5-FU to overcome multidrug resistance, the nanobeacons' intracellular uptake and trafficking was examined using a 5-FU resistant MDA-MB-231 breast cancer cell line obtained by continuous culturing of parental MDA-MB-231 cells in 0.05 mg/mL dose of 5-FU. As explained below, drug resistance was confirmed by the absence of cell death using MTT assay after continuous exposure to drug, when compared to parental cancer cells, as shown at FIG. 6.

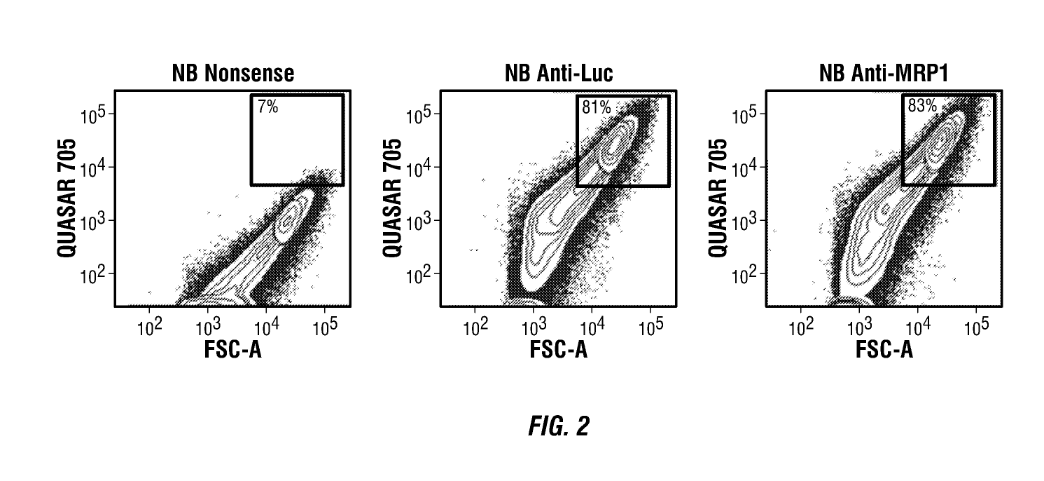

Dark-gold nanobeacons entered and accumulated in resistant MDA-MB-231 cells as verified by confocal microscopy demonstrating specific targeting at 48 hours after incubation of anti-Luc and anti-MRP1 nanobeacons when compared to nanobeacon nonsense. It was evident that silencing occurred only with the anti-Luc and anti-MRP1 nanobeacons upon hairpin conformational change due to specific hybridization to the target sequence, which was identified via fluorescence emission. Epi-fluorescence images of [1] cellular uptake kinetics of cells incubated with anti-MRP1 nanobeacons for 0.5, 2, 4, 8, 24, and 48 hours, and [2] nanobeacons' cellular uptake and detection associated with drug release at the same time points demonstrated the foregoing. And, as depicted at FIG. 2, analytical flow cytometry data confirmed the confocal images, where a substantial increase in Q705 intensity is observed only for nanobeacons anti-Luc and anti-MRP1 when compared to nanobeacon nonsense. Confocal images of breast cancer cells further revealed that the dark-gold nanobeacons anti-MRP1 were co-localized in intracellular organelles, such as endosomes and/or lysosomes at 24 hours and that these dark-gold nanobeacons could escape these organelles at 48 hours.

In this example, MDA-MB-231 cells (from triple-negative breast cancer) were grown in Dulbecco's modified Eagle's medium (DMEM, Invitrogen, Carlsbad, Calif., USA) supplemented with 4 mM glutamine, 10% heat inactivated fetal bovine serum (Invitrogen, Carlsbad, Calif., USA), 100 U/mL penicillin, and 100 μg/mL streptomycin (Invitrogen, Carlsbad, Calif., USA), and maintained at 37° C. in 5% CO₂.

Not wishing to be bound by any particular theory, breast cancer was chosen as a model since it can benefit from local noninvasive administration of an injectable hydrogel. Moreover, TNBC accounts for 15% to 20% of all breast cancers, and it represents the most aggressive subtype with a direct prognosis. TNBCs also are characterized by resistance to apoptosis, aggressive cellular proliferation, migration and invasion, and currently lack molecular markers and effective targeted therapy. TNBC patients, therefore, frequently suffer from poor survival rates and limited efficacy of neoadjuvant chemotherapy, as tumors from such patients are characterized by overexpression of specific genes involved in drug desensitizing mechanisms.

Cells were seeded at a density of 1×10⁵ cells/well in 24-well plates and grown for 24 hours prior to incubation of nanobeacons. On the day of incubation, the cells were approximately 50% confluent. A multidrug resistant breast cancer cell line MDA-MB-231 was also developed by continuous culture of parental MDA-MB-231 cells in 5-FU and maintained with 5-FU at a dose of 0.05 mg/mL for 6 weeks.

After 6 weeks, the drug resistance was checked by the absence of cell death via an MTT assay after continuous exposure to drug, when compared to parental cancer cells. Briefly, a standard MTT [3-(4,5-dimethylthiazol-2-yl)-2,5-diphenyltetrazoliumbromide] reduction assay was performed to determine the resistance status of the cells and the cytotoxicity of the gold nanobeacons.

Cells were seeded at a density of 1×10⁵ cells per well in 24-well culture plates in complete DMEM (500 μL) with serum. After 24 hours of exposure, the medium was removed and the cells were washed twice with sterile PBS and 300 μL of fresh medium with serum was added. Then 16.7 μL of sterile MTT stock solution (5 mg/mL in PBS) was added to each well.

After incubation for an additional 2 hours, the medium was removed and the formazan crystals were resuspended in 300 μL of dimethyl sulfoxide (Sigma-Aldrich, USA). The solution was mixed and its absorbance was measured at 540 nm as a working wavelength and 630 nm as reference using a microplate reader (Varioskan Flash Multimode Reader, Thermo Scientific, MA, USA). The cell viability was normalized to that of cells cultured in the culture medium with PBS treatment.

The viability of cells was also visualized by using a double staining procedure with acridine orange (AO, green, live cells) and propidium iodide (PI, red, dead cells). Briefly, 0.5 mL of complete medium containing 0.67 μM AO and 75 μM PI was added to each well and was incubated in the dark at 37° C. for 30 minutes. After rinsing with fresh medium, live and dead cells were monitored by using a fluorescence microscope (Nikon Eclipse Ti). For confocal microscopy, cells were fixed with 4% paraformaldehyde in PBS for 15 minutes at 37° C. and mounted in ProLong® Gold Antifade Reagent with DAPI (Invitrogen, Carlsbad, Calif., USA) to allow for nuclear staining. Images of cells were taken with a Nikon AIR Ultra-Fast Spectral Scanning Confocal Microscope.

MDA-MB-231 cells incubated with Q705 dark gold nanobeacons were analyzed and data were acquired on FACS LSR HTS-2 (BD Biosciences, NJ, USA) flow cytometer.

In order to corroborate confocal and flow cytometry data and overcome 5-FU resistance, resistant MDA-MB-231 cells were incubated with increasing amounts of dark-gold nanobeacons with and without 5-FU, and evaluated by the imaging system IVIS®. As expected, only anti-Luc and anti-MRP1 nanobeacons loaded with 5-FU produced a significant decrease in luminescence. The same was observed for cell viability evaluated by MTT assay. Also, dark-gold nanobeacons without 5-FU did not affect cell viability, when compared to dark-gold nanobeacons loaded with 5-FU, with an IC50 near 0.5 nM at 72 hours of exposure.

Live-dead staining of resistant MDA-MB-231 cells after uptake of increasing concentrations (0.1, 1, and 5 nM) of dark-gold nanobeacons for 48 hours was performed using double staining procedure with acridine orange (AO) and propidium iodide (PI) representing green and red fluorescence for live and dead cells, respectively. The test revealed that only anti-MRP1 nanobeacons loaded with 5-FU (at 5 nM) resulted in extensive cell death (>95%). This was believed, at least in this example, to be due to MRP1 inhibition and consequent release of 5-FU.

Anti-Luc nanobeacons demonstrated modest cell death despite releasing the drug without MRP1 inhibition, as shown at FIG. 8. It was believed that this occurred after MRP1 was able to actively pump cytotoxic drugs like 5-FU from the cytosol across the plasma membrane at the expense of ATP. At FIG. 8, the silencing effect was expressed as a concentration-dependent decrease in MRP1 relative expression. Only anti-MRP1 nanobeacons loaded with 5-FU showed potent silencing with an ED50 around 1 nM for 48 hours of incubation. The data points of FIG. 8 represent group mean±SD (n=3), and GAPDH was used as the reference gene.

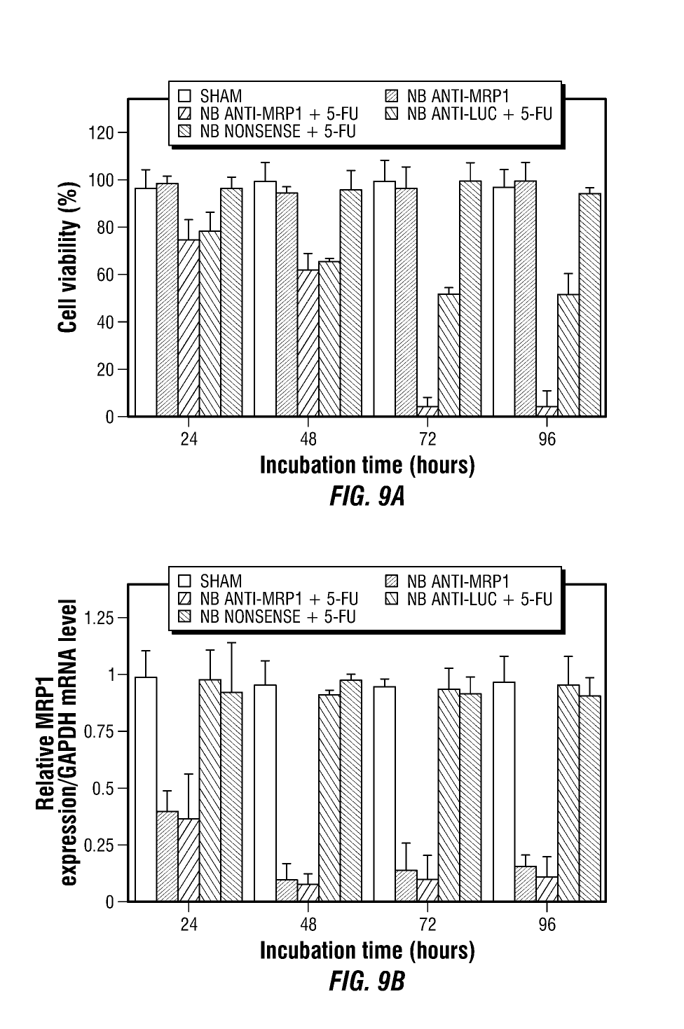

In order to evaluate the kinetics of the 5-FU effect and the mRNA MRP1 knockdown by the nanobeacons, an MTT cell viability assay was performed after 24, 48, and 72 hours, and compared with the kinetics for MRP1 expression using 5 nM (the same concentration for in vitro and in vivo studies) of nanobeacons loaded with 5-FU, as showed at FIG. 9A and FIG. 9B. These data demonstrated, for this example, that maximum mRNA knockdown (at 48 hours) occurred 24 hours before the maximum effect of the drug (at 72 hours), which showed that the 5-FU was mostly effective 24 hours after the drug reversal mechanism took place in this example.

### Example 6

### Quantitative PCR

Total RNA from MDA-MB-231 cells and breast tumors from SCID mice was extracted using RNeasy Plus Mini Kit (Qiagen, Hilden, Germany) according to the manufacturer's protocol. cDNA was produced using a High-Capacity cDNA Reverse Transcription Kit (Applied Biosystems, CA, USA) using 500 ng of total RNA. qRT-PCR was performed with TaqMan® probes FAM-MGB for MRP1 (ABCC1) and GAPDH (Applied Biosystems, CA, USA). GAPDH was used as a reference gene.

The reactions were processed using Light Cycler 480 II Real-time PCR machine (Roche, Switzerland) using TaqMan® Gene Expression Master Mix (Applied Biosystems, CA, USA) under the following cycling steps: 2 minutes at 50° C. for UNG activation; 10 minutes at 95° C.; 40 cycles at 95° C. for 15 seconds; and 60° C. for 60 seconds. At least three independent repeats for each experiment were carried out. Gene expression was determined as a difference in fold after normalizing to the housekeeping gene GAPDH.

### Example 7

### Development of Orthotopic Triple-Negative Breast Cancer Mice Model

In order to evaluate the efficiency of the dark-gold nanobeacon probes in sensing and in overcoming MDR in vivo, an orthotopic breast cancer mice model was developed by injecting resistant MDA-MD-231 cells to the mammary fat pad of female SCID hairless congenic mice. Efficacious and local delivery of the dark-gold nanobeacon probes was achieved by the implantation of a hydrogel disk on top of the triple-negative breast tumors. The hydrogel scaffold comprising polyamidoamine (PAMAM G5) dendrimer cross-linked with dextran aldehyde provided enhanced stability of the embedded nanoparticles. Epi-fluorescence images showed a homogeneous distribution of the dark-gold nanobeacon probes (previously hybridized with a complementary target) in the hydrogel network. The dual labeling disclosed a co-localization of the nanobeacon probes and the tagged polymeric matrix that may be attributed to electrostatic interaction between the nanobeacons and the hydrogel, and the release profile of the nanobeacons from the dendrimer:dextran scaffold during 96 hours revealed an almost complete release in the first 24 hours under physiological conditions in vitro (pH 7.4 and 37° C.).

The reversible interaction was believed to enable rapid local release of the nanoparticles at the site of interest, as demonstrate by the almost complete release in the first 24 hours under physiological conditions in vitro (pH 7.4 and 37° C.) without altering the nanobeacons' stability in the presence of the dextran and dendrimer components of the hydrogel (see Example 10). Tumors in the mammary fat pad were induced in female SCID hairless congenic mice by injection of 5×106 resistant MDA-MB-231 cells stably expressing firefly luciferase, suspended in 50 μL of HBBS (Lonza, GA, USA) solution. For determination of tumor growth, individual tumors were measured using caliper, and tumor volume was calculated by Tumor volume (mm³)=width×(length²)/2. Treatments began when tumor volume reached about 100 mm³.

### Example 8

### Hydrogel-Dark-Gold Nanobeacons Synthesis and In Vivo Implantation

Dextran aldehyde was tagged by reaction with fluorescein thiosemicarbazide (Invitrogen, Carlsbad, Calif., USA) in 20 mL of 0.1 M phosphate buffer (pH 7.5) for 30 minutes at room temperature. The reaction crude was then cooled down in an ice water bath, and imine bonds were reduced with 20 mL of 30 mM sodium cyanoborohydrate in PBS for 30 minutes.

Tagged dextran aldehyde was then dialyzed against double distilled water using a 10,000 Da molecular cut-off filter for 8 days and then lyophilized. Tagged hydrogel scaffolds were developed as known in the art (e.g., Oliva, N. et al. LANGMUIR 2012, 28, 15402-15409).

Equal parts of PAMAM G5 dendrimer (Dendriteck, Inc., MI, USA) amine of 12.5% solid content and dextran aldehyde 5% solid content with 0.25% fluorescently labeled dextran were mixed to form 6 mm pre-cured disks. For doped scaffolds, nanobeacons were added to the dendrimer solution prior to hydrogel formation at a concentration of 20 nM. All solutions were filtered through a 0.22 μm filter prior to hydrogel formation for in vivo implantation.

Pre-cured disks of fluorescently labeled scaffold with or without nanobeacons were formed and implanted subcutaneously on top of the fat mammary tumor in SCID mice. Specifically, once tumors reached the desired volume of about 100 mm³, hydrogel scaffolds loaded with dark-gold nanobeacons were implanted adjacent to the mammary fat pad tumor.

### Example 9

### Hydrogel-Dark-Gold Nanobeacons Fluorescence Images

Pre-cured fluorescently labeled scaffolds alone (control), doped with non-hybridized nanobeacons, and doped with hybridized nanobeacons were snap-frozen in liquid nitrogen and kept at −80° C. for 24 hours. Then, 12 μm thick cryosections (Cryostat Leica CM1850, Leica, IL, USA) were analyzed by fluorescence microscopy (NIS-Elements Nikon, Tokyo, JP).

### Example 10

### Dark-Gold Nanobeacons Stability In Vitro

A stability assay for nanobeacons with and without target in the presence of dextran 5% and dendrimer 12.5% during 24 hours revealed that the nanobeacons were stable at physiological conditions.

Nanobeacons (final concentration 5 nM) were pre-treated with and without complementary target MRP1, and incubated at 37° C. in dextran and dendrimer solutions (final concentrations of 5% and 12.5% in water, respectively). Samples were collected at different time points and fluorescence measured (Varioskan Flash Multimode Reader, Thermo Scientific, MA, USA). Fluorescence intensity was plotted over time.

### Example 11

### Hydrogel-Dark-Gold Nanobeacons Release In Vitro

Pre-cured disks of fluorescently labeled hydrogel scaffold doped with hybridized nanoparticles were incubated in PBS at 37° C. At different time points, samples were collected from the PBS and fluorescence of the released products was quantified (Varioskan Flash Multimode Reader, Thermo Scientific, MA, USA). Data were plotted as the percent of nanobeacons/dextran aldehyde released for each time point. Controls for this experiment included a scaffold without nanobeacons, and a scaffold with non-hybridized nanobeacons.

### Example 12

### Analysis of Tumor Growth and Hydrogel-Gold Nanobeacon Degradation

Non-invasive longitudinal monitoring of tumor progression was followed by scanning mice with the IVIS Spectrum-bioluminescent and fluorescent imaging system (Xenogen XPM-2 Corporation, CA, USA) from mice bearing mammary tumors from MDA-MB-231 cells (n=5 animals per treated group). Fifteen minutes before imaging, mice were administered with 150 pt of D-luciferin (30 mg/mL, Perkin Elmer, GA, USA) in DPBS via intraperitoneal injection. Whole-animal imaging was performed at the indicated time points—days 0 (2 hours after gel implantation), 1, 2, 4, 7, and 14. Assessment of in vivo toxicity via mice body weight evaluation was performed on all the animal groups during 37 days after hydrogel-gold-nanobeacons exposure.

In vivo imaging was used to track simultaneously tumor inhibition as measured by luciferase expression, nanobeacon probes before and after hybridization to the target and hydrogel stability monitored by fluorescence emission over a period of 14 days following hydrogel implantation.

No signs of inflammation were observed at the surgical site and no changes in body weight followed hydrogel implantation, which seemed to suggest that the hydrogels were biocompatible, with associated toxicity or side effects.

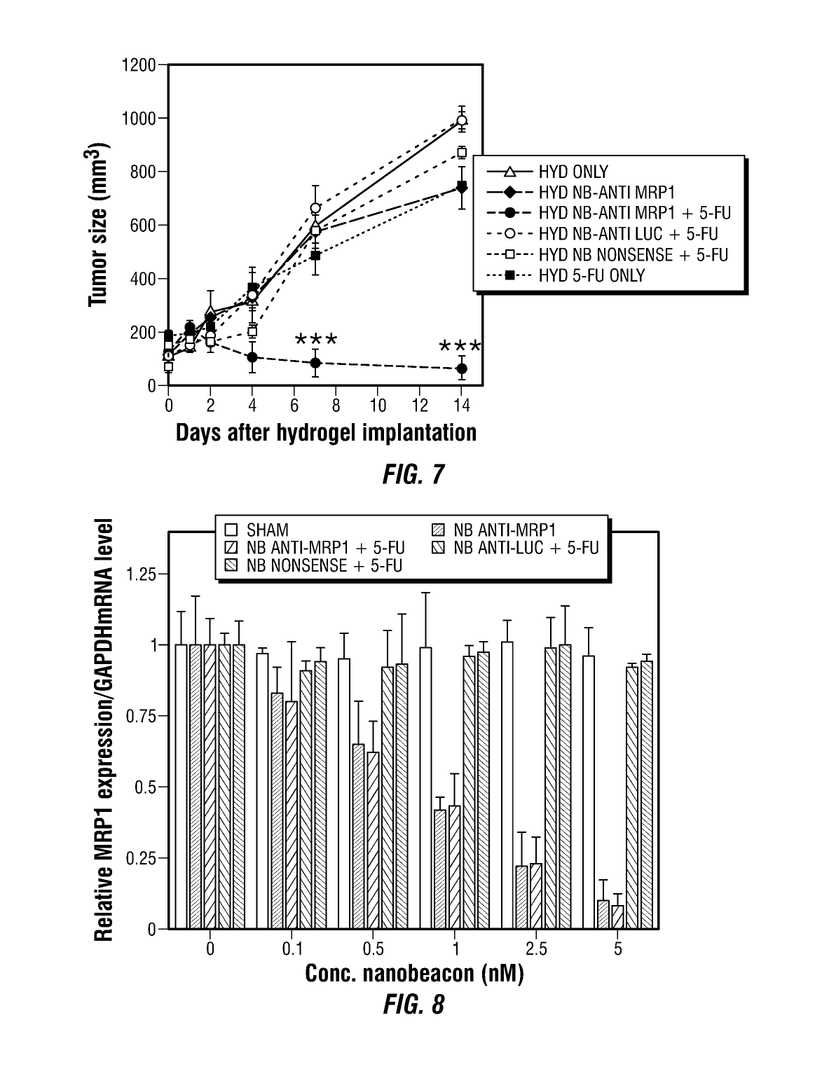

Bioluminescence imaging of mice revealed that only anti-MRP1 nanobeacons loaded with 5-FU promoted efficient and sustained inhibition of tumor progression, with an approximately 90% tumor size reduction 14 days after hydrogel disk implantation. Representative images of whole body organs and resected tumors in mice treated with nanobeacons corroborated the results regarding tumor size reduction after hydrogel implantation (see FIG. 7). Therefore, despite the cross-resistance to 5-FU, over 90% tumor reduction was achieved in vivo in the triple negative breast model following 80% MRP1 silencing, compared to the continuous tumor growth following only drug or inactive nanoprobe administration.

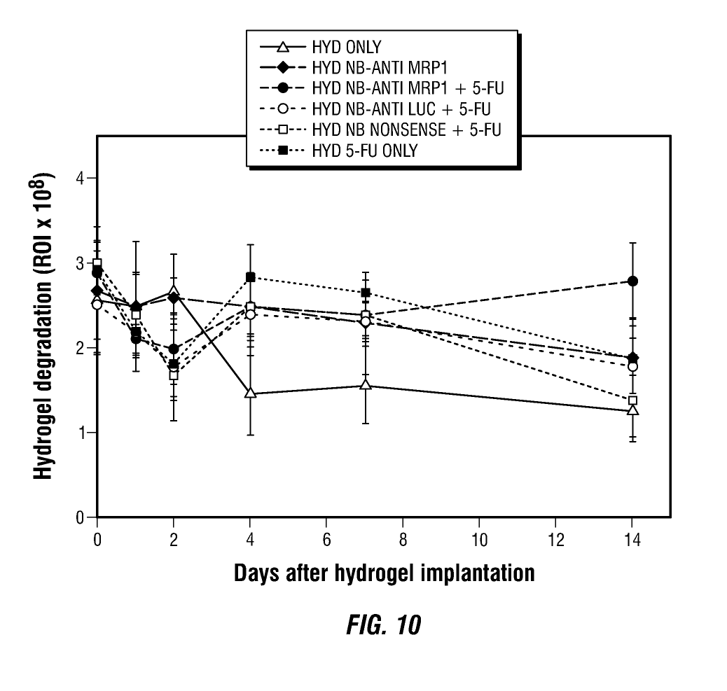

Also, in vivo hydrogel degradation monitored by FITC intensity following implantation in mice, as shown at FIG. 10, coincided with nanobeacon fluorescence. The strongest signals were observed during the first 2 days in which the hydrogel degrades rapidly. The hydrogel continued to degrade about 45-50% after 14 days.

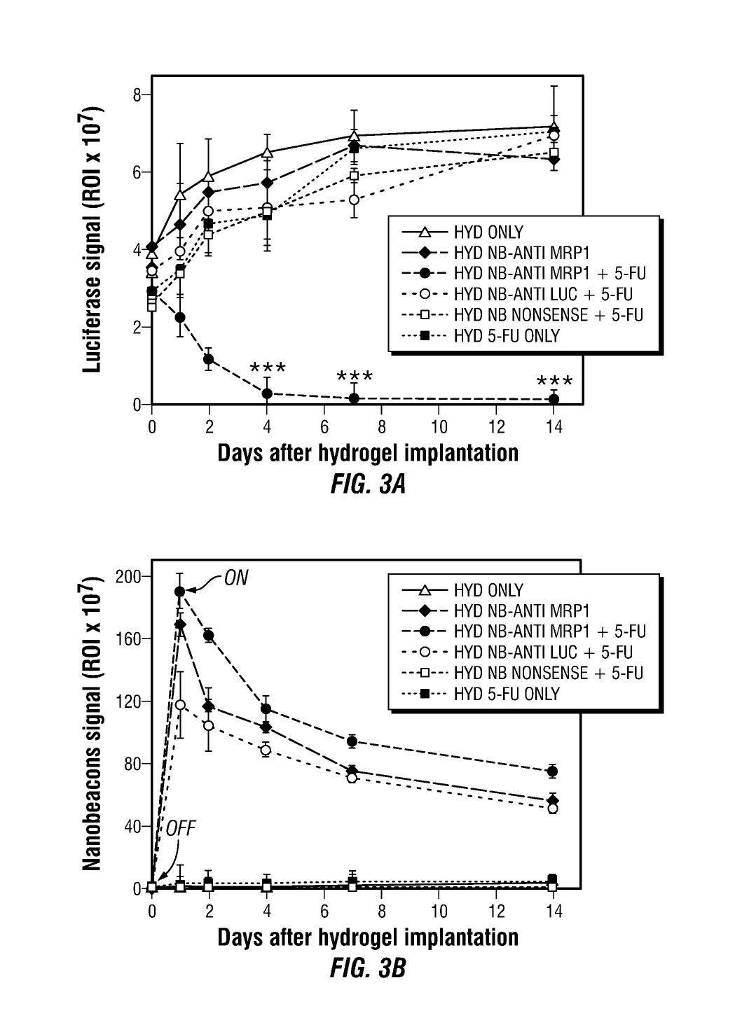

Monitoring the change in tumor size as a function of time after treatment with nanobeacons revealed a significant reduction in tumor growth (n=5, P<0.005) at day 14 post treatment, as depicted at FIG. 3A, with an almost complete tumor regression only for the anti-MRP1 nanobeacons loaded with 5-FU group. Regarding FIG. 3A, an approximately 90% decrease in luciferase activity an in tumor size was observed exclusively for the anti-MRP1 nanobeacons loaded with 5-FU treated tumors compared to free drug (5-FU only), anti-MRP1 only, anti-Luc loaded with 5-FU, and nonsense nanobeacons with 5-FU (n=5, P<0.005).

The free drug had no effect, as expected, once the cells were resistant to 5-FU, and only when MRP1 was silenced with the anti-MRP1 nanobeacons did the drug (5-FU) once again become effective.

Concerning nanobeacon probes imaging, as shown at FIG. 3B, fluorescence images of implanted hydrogels revealed, as expected, that the fluorescence signal was “off” at day 0 (2 hours after surgery) and was turned “on” at day 1 (24 hours), reaching a maximum intensity for MRP1 and luciferase detection at day 2 (48 hours) for anti-MRP1 nanobeacons with and without 5-FU, and anti-Luc nanobeacons loaded with 5-FU.

### Example 13

### Ex Vivo Tumor Characterization and Biodistribution

Ex vivo organ biodistribution of nanobeacons at day 14 showed remarkable accumulation in the tumors of the previous example, with no unspecific accumulation in the other main organs. In fact, approximately 40% of the nanobeacon probes persisted in the tumoral tissue. The data confirmed the specificity of this platform in vivo, not only for sensing but specifically to overcome MDR in breast tumors.

To confirm the observed in vivo effects, gene expression analysis of resected tumors was conducted to verify silencing of MRP1, as silencing of an important MDR gene would have imparted substantially higher intracellular concentrations of the chemotherapeutic drug. Indeed, real-time PCR results showed a decrease in MRP1 expression in both anti-MRP1 nanobeacons loaded with 5-FU and without 5-FU.

MRP1 is a cell-surface efflux pump involved in the redox regulation of MDR by reducing the intracellular concentration of 5-FU, which makes MRP1 a clinically relevant biomarker for triple-negative breast cancer; nevertheless, expression of VEGF and EGFR decreased only after treatment with anti-MRP1 nanobeacons loaded with 5-FU. H&E staining of breast tumor sections showed evidence of extensive reduction in vascularization only for anti-MRP1 nanobeacons loaded with 5-FU, in accordance with tumor size reduction due to overcoming drug resistance. Immunohistochemical analysis showed that the expression of VEGF and the number of microvessels was reduced only after treatment with hydrogels embedded with anti-MRP1 nanobeacons with 5-FU. Increasing number of large vessels was found in the control groups, when compared to anti-MRP1 nanobeacons with 5-FU.

We claim:

1. A theranostic nanoprobe comprising:
a gold nanoparticle functionalized with
at least one DNA-hairpin labeled with a fluorophore, and
at least one anchor labeled with a quencher; and
a chemotherapeutic agent intercalated in the at least one DNA-hairpin;
wherein the at least one DNA-hairpin is configured to hybridize to a complementary target in a cancer cell.

2. The theranostic nanoprobe of claim 1, wherein the gold nanoparticle is further functionalized with a spacer comprising PEG.

3. The theranostic nanoprobe of claim 2, wherein the spacer comprising PEG is derived from α-Mercapto-ω-carboxy PEG.

4. The theranostic nanoprobe of claim 2, wherein from about 20% to about 40% of the surface area of the gold nanoparticle is functionalized with the spacer comprising PEG.

5. The theranostic nanoprobe of claim 1, wherein the average diameter of the gold nanoparticle is from about 10 to about 16 nm.

6. The theranostic nanoprobe of claim 1, wherein the DNA-hairpin is a thio-DNA-hairpin.

7. The theranostic nanoprobe of claim 1, wherein the anchor is a thio-DNA oligonucleotide.

8. The theranostic nanoprobe of claim 1, wherein the ratio of DNA-hairpin:gold nanoparticle is from about 20:1 to about 40:1.

9. The theranostic nanoprobe of claim 1, wherein the fluorophore is a near-infrared dye.

10. The theranostic nanoprobe of claim 9, wherein the near-infrared dye comprises Quasar® 705 dye.

11. The theranostic nanoprobe of claim 1, wherein the quencher is Black Hole® Quencher 2.

12. The theranostic nanoprobe of claim 1, wherein the complementary target is MRP1 mRNA.

13. The theranostic nanoprobe of claim 1, wherein the chemotherapeutic agent is selected from 5-fluorouracil, cisplatin, doxorubicin, rebeccamycin, epirubicin, mitoxantrone, tomaymycin, anthramycin, carboplatin, oxaliplatin, satraplatin, picoplatin, nedaplatin, triplatin, gemcitabine, vincristine, or a combination thereof.

14. The theranostic nanoprobe of claim 1, wherein the gold nanoparticle at least partially quenches the emission of the chemotherapeutic agent.

15. The theranostic nanoprobe of claim 1, further comprising a hydrogel in which the gold nanoparticle is embedded.

16. The theranostic nanoprobe of claim 15, wherein the hydrogel comprises a dendrimer and a polymer.

17. The theranostic nanoprobe of claim 16, wherein the dendrimer is a G5 PAMAM-derived dendrimer having primary amines on about 25% of the dendrimer's surface groups.

18. The theranostic nanoprobe of claim 16, wherein the polymer is dextran with a molecular weight of about 10 kDa and about 50% of its hydroxyl groups converted to aldehydes.

19. A method for treating a biological tissue, the method comprising:
providing a hydrogel comprising an embedded theranostic nanoprobe; and
contacting the biological tissue with the hydrogel; wherein the embedded theranostic nanoprobe comprises
a gold nanoparticle functionalized with
at least one DNA-hairpin labeled with a fluorophore, and
at least one anchor labeled with a quencher; and
a chemotherapeutic agent intercalated in the at least one DNA-hairpin;
wherein the at least one DNA-hairpin is configured to hybridize to a complementary target in a cancer cell.

20. The method of claim 19, wherein the biological tissue comprises a tumor.

21. The method of claim 19, wherein contacting the biological tissue with the hydrogel comprises applying the hydrogel on a surface of the biological tissue.

22. The method of claim 19, wherein contacting the biological tissue with the hydrogel comprises injecting the hydrogel into the biological tissue.

23. A method for treating a biological tissue, the method comprising:
providing a first solution comprising a polymer component, wherein the polymer component comprises a polymer having three or more aldehyde groups;
providing a second solution comprising a dendrimer component, wherein the dendrimer component comprises a dendrimer having at least 2 branches with one or more surface groups;
wherein at least one of the first solution and the second solution comprises a theranostic nanoprobe; and
combining the first and second solutions together to produce a hydrogel and contacting one or more biological tissues with the hydrogel;
wherein the embedded theranostic nanoprobe comprises
a gold nanoparticle functionalized with
at least one DNA-hairpin labeled with a fluorophore, and
at least one anchor labeled with a quencher; and
a chemotherapeutic agent intercalated in the at least one DNA-hairpin;
wherein the at least one DNA-hairpin is configured to hybridize to a complementary target in a cancer cell.

24. The method of claim 23, wherein the biological tissue comprises a tumor.

25. The method of claim 23, wherein contacting the biological tissue with the hydrogel comprises applying the hydrogel on a surface of the biological tissue.

26. The method of claim 23, wherein contacting the biological tissue with the hydrogel comprises injecting the hydrogel into the biological tissue.

27. A hydrogel composition comprising:
a plurality of theranostic nanoprobes, each of which comprises
a gold nanoparticle functionalized with
at least one DNA-hairpin labeled with a fluorophore, and
at least one anchor labeled with a quencher; and
a chemotherapeutic agent intercalated in the at least one DNA-hairpin,
wherein the at least one DNA-hairpin is configured to hybridize to a complementary target in a cancer cell; and
a biocompatible hydrogel in which the plurality of theranostic nanoprobes are dispersed.

28. A kit comprising:
a first solution comprising a polymer component, wherein the polymer component comprises a polymer having three or more aldehyde groups;
a second solution comprising a dendrimer component, wherein the dendrimer component comprises a dendrimer having at least 2 branches with one or more surface groups; and
a theranostic nanoprobe dispersed in either the first solution, the second solution, or both, wherein the theranostic nanoprobe comprises
a gold nanoparticle functionalized with
at least one DNA-hairpin labeled with a fluorophore, and
at least one anchor labeled with a quencher; and
a chemotherapeutic agent intercalated in the at least one DNA-hairpin;
wherein the at least one DNA-hairpin is configured to hybridize to a complementary target in a cancer cell.

29. A theranostic nanoprobe comprising:
a gold nanoparticle functionalized with at least one DNA-hairpin; and
a chemotherapeutic agent intercalated in the at least one DNA-hairpin;
wherein the at least one DNA-hairpin is configured to hybridize to a complementary target in a cancer cell.

30. The theranostic nanoprobe of claim 29, wherein the complementary target is MRP1 mRNA.

31. The theranostic nanoprobe of claim 29, further comprising a hydrogel in which the gold nanoparticle is embedded.

32. The theranostic nanoprobe of claim 31, wherein the hydrogel comprises a G5 PAMAM-derived dendrimer having primary amines on about 25% of its surface groups.

33. The theranostic nanoprobe of claim 31, wherein the hydrogel comprises dextran with a molecular weight of about 10 kDa and about 50% of its hydroxyl groups converted to aldehydes.
